(19) United States
(12) Patent Application Publication
(10) Pub. No.: US 2018/0338922 A1
(43) Pub. Date: Nov. 29, 2018

(54) FIXED DOSE FORMULATIONS

(71) Applicant: Esperion Therapeutics, Inc., Ann Arbor, MI (US)

(72) Inventors: Mohamed Abdelnasser, New York, NY (US); Pratibha S. Pilgaonkar, Mumbai (IN); Anilkumar S. Gandhi, Mumbai (IN)

(21) Appl. No.: 15/859,279

(22) Filed: Dec. 29, 2017

Related U.S. Application Data
(60) Provisional application No. 62/511,889, filed on May 26, 2017.

(51) Int. Cl. A61K 9/24; A61K 31/20; A61K 31/397; A61K 9/28; A61K 9/20

(52) U.S. Cl. CPC A61K 9/209; A61K 31/20; A61K 31/397; A61K 9/284; A61K 9/282; A61K 9/2018; A61K 9/2009; A61K 9/2013; A61K 9/2054; A61K 9/2027; A61K 9/2813

(57) ABSTRACT

Herein disclosed are novel compositions comprising: Bempedoic acid and Bempedoic acid and Ezetimibe, kits, methods of using and processes for making said novel compositions. Notably, the formulations herein provide pharmaceutical compositions having excellent stability and release properties for both drug products. These improved formulations are useful in the treatment and prevention of cardiovascular disease.

### CROSS-REFERENCE TO RELATED APPLICATIONS

This application claims the benefit of U.S. Provisional Application No. 62/511,889 filed on May 26, 2017 and is hereby incorporated by reference in its entirety.

### BACKGROUND OF THE INVENTION

### Field of the Invention

The present disclosure relates to formulations, kits, methods of use and methods for making pharmaceutical formulations comprising Bempedoic acid and Ezetimibe.

### Description of the Related Art

Certain therapeutic molecules belonging to categories or chemical classes have been identified, or rather acknowledged to possess poor flow and sticky bulk properties. Further, although not a formally expressed rule, chemists generally observe that compounds from Biopharmaceutical Classification System (BCS) class II are difficult to formulate owing to the fact that class II compounds are poorly water soluble and hence, suffer from poor dissolution in the gastrointestinal tract. Both Bempedoic acid (ETC-1002) and Ezetimibe are in the drug product BCS Class II compounds. Both are poorly soluble in water and highly permeable. In the solid state, Bempedoic acid exhibits poor flow characteristics and is very sticky. The observed stickiness adversely impacts various stages during development of pharmaceutical formulations including weighing, blending, granulation and compression. These problems adversely impact drug manufacturing operations, notably tablet compression (low rpm operation, weight variation, frequent machine stoppage; etc.). Standard granulation of Bempedoic acid only marginally reduces the sticky behavior thereby improving processability. Bempedoic acid also has a relatively low melting point, 88-91° C., and as such contributes to the diminished plasticity of the bulk.

Formulation chemists have provided solutions; however, such work is unique to the particular drug being studied. A balance must be struck between stability and release characteristics such that adapted flow and other bulk physical properties meet pre-defined safety requirements for each API. This makes the art of API formulation very unpredictable. Thus, formulation chemists do not have a single universal set of rules or additives that enhance any given API's pharmacodynamic and/or bulk properties.

Accordingly, there is a need to develop stable and effective pharmaceutical compositions that allow for a formulation of Bempedoic acid and Ezetimibe hat have improved, desirable PK and bulk physical properties.

The present disclosure overcomes difficulties associated with co-formulating Bempedoic acid and Ezetimibe, as described in detail below.

### SUMMARY

Herein disclosed is the development of novel combination formulations for a drug product comprising Bempedoic acid (ETC-1002) and Ezetimibe. Also disclosed herein are granulated compositions of Bempedoic acid.

Two formulation options for the combination are identified as improved and compatible for both Bempedoic acid (ETC-1002) and Ezetimibe from a bioavailability study conducted and disclosed herein: a monolayer and a bilayer tablet formulation. The monolayer tablet is manufactured with granulated mixtures from both compounds blended together into a single layer. The bilayer tablet is manufactured with granulated mixtures from each compound compressed into two (2) separate layers.

The inventors have found that surface treatment of ETC-1002 with colloidal silicon dioxide reduces or eliminates the stickiness problem. This treatment involves blending ETC-1002 with colloidal silicon dioxide first, and then mixing the blend with hydroxypropyl cellulose (HPC-L) and microcrystalline cellulose in rapid mixer granulator; prior to granulation. Granulation is also carried with a binder solution comprising colloidal silicon dioxide and hydroxypropyl cellulose (HPC-L). The treatment of ETC-1002 and the preparation of the premix for granulation is carried out in such a way that: 1) excessive hydrophobicity is not imparted to the active, 2) the dissolution and release profile of ETC-1002 is not adversely impacted, 3) the stability of ETC-1002 is not adversely affected, and 4) incompatibility from any of the excipients does not arise in the fixed dose combination formulation containing Ezetimibe, particularly in the monolayer formulation.

### BRIEF DESCRIPTION OF THE DRAWINGS

FIG. 1 shows the Zetia dissolution profile for Ezetimibe in 500 mL of dissolution medium using a USP Apparatus-II at 50 rpm.

FIG. 2 shows the dissolution profile of Bempedoic Acid tablet in different media with 2.0% w/v sodium lauryl sulfate (SLS).

FIG. 3 depicts the dissolution profile for Reference Product combinations at various dissolution conditions.

FIG. 4 is a graph of the dissolution profiles of the Reference Product and Fixed Dose Combination product in discriminatory dissolution media reflecting the difference in granulation processing.

FIG. 5 shows the dissolution profile of Bempedoic Acid in three different discriminatory media.

FIG. 6 is a graph showing the comparative dissolution profile of Bempedoic Acid Tablet (the Reference Product) vs. Fixed Dose Combination-monolayer tablets having Coarse and Fine Grade Bempedoic Acid.

FIG. 7 depicts a surface treatment of the granulated particles having Bempedoic Acid with Aerosil® and HPC-L binder.

FIG. 8 is a graph showing the dissolution profile of tablets having different binder concentrations.

FIG. 9 depicts the comparative dissolution profile of a prototype Fixed Dose Combination tablet and a test Ezetimibe tablet (10 mg) in discriminatory dissolution media.

FIG. 10 depicts the comparative dissolution profile for different batches of granulated Ezetimibe.

FIG. 11 illustrates the Fixed Dose Combination-monolayer tablet manufacturing process.

FIG. 12 illustrates the Fixed Dose Combination-bilayer tablet manufacturing process.

FIG. 13 shows comparative dissolution profiles for Ezetimibe from the monolayer and bilayer tablets against a Reference Product.

FIG. 14 shows comparative dissolution profiles of Bempedoic acid from the monolayer and bilayer tablets against a Reference Product.

FIG. 15 shows comparative dissolution profiles of Ezetimibe from the Fixed Dose Combination-monolayer tablet against an Ezetimibe test product.

FIG. 16 and FIG. 17 show comparative dissolution profiles of Bempedoic acid from the monolayer and bilayer tablets against an Ezetimibe test product.

FIG. 18 shows comparative dissolution profiles of Bempedoic acid from the Fixed Dose Combination test product in QC media.

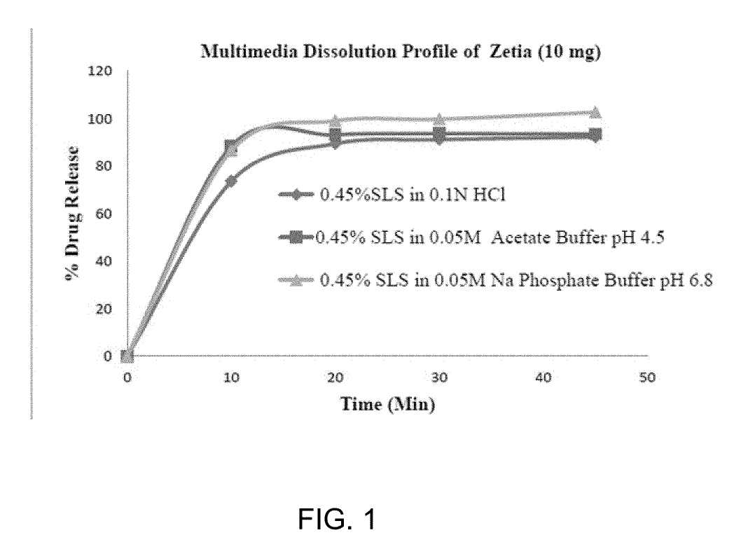

### DETAILED DESCRIPTION

### Advantages and Utility

Briefly, and as described in more detail below, described herein are novel compositions containing Bempedoic acid or Bempedoic acid and Ezetimibe, kits, methods of using and processes for making said compositions. The advantages for this approach are numerous and include, but are not limited to, improved pharmacokinetic (PK) properties of one or both of Bempedoic acid and Ezetimibe, and improved flowability and other bulk physiochemical properties of the composition in the solid state. As described above, many BCS class II compounds suffer from diminished PK and bulk properties. Hence, there is a significant need for formulations that improve the physiochemical properties of pharmaceutical compositions containing Bempedoic acid.

### Definitions

Terms used in the claims and specification are defined as set forth below unless otherwise specified. Further, if any term or symbol used herein is not defined as set forth below, it shall have its ordinary meaning in the art.

The practice of the present invention includes the use of conventional techniques of organic chemistry, molecular biology (including recombinant techniques), microbiology, cell biology, biochemistry and immunology, which are within the skill of the art.

As used herein and in the appended claims, singular articles such as “a,” “an” and “the” and similar referents in the context of describing the elements (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, including the upper and lower bounds of the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the embodiments and does not pose a limitation on the scope of the claims unless otherwise stated. No language in the specification should be construed as indicating any non-claimed element as essential.

As used herein, the term “cardiovascular diseases” refers to diseases of the heart and circulatory system. These diseases are often associated with dyslipoproteinemias and/or dyslipidemias. Cardiovascular diseases which the compositions of the present invention are useful for preventing or treating include but are not limited to arteriosclerosis; atherosclerosis; stroke; ischemia; endothelium dysfunctions, in particular those dysfunctions affecting blood vessel elasticity; peripheral vascular disease; coronary heart disease; myocardial infarction; cerebral infarction and restenosis.

As used herein, the term “dyslipidemias” refers to disorders that lead to or are manifested by aberrant levels of circulating lipids. To the extent that levels of lipids in the blood are too high, the compositions of the invention are administered to a patient to restore normal levels. Normal levels of lipids are reported in medical treatises known to those of skill in the art. For example, recommended blood levels of LDL, HDL, free triglycerides and others parameters relating to lipid metabolism can be found at the web site of the American Heart Association and that of the National Cholesterol Education Program of the National Heart, Lung and Blood Institute (http://www.americanheart.org/cholesterol-/about_level.html and http://www.nhlbi.nih.gov/health/public/heart/chol/hb-c_what.html, respectively). At the present time, the recommended level of HDL cholesterol in the blood is above 35 mg/dL; the recommended level of LDL cholesterol in the blood is below 130 mg/dL; the recommended LDL:HDL cholesterol ratio in the blood is below 5:1, ideally 3.5:1; and the recommended level of free triglycerides in the blood is less than 200 mg/dL.

The term “subject” refers to any mammal including humans, and so includes mammals such as those animals of veterinary and research interest that are including, but not limited to: simians, cattle, horses, dogs, cats, and rodents. The term “subject” is interchangeable with the term “patient”.

The term “sufficient amount” means an amount sufficient to produce a desired effect, e.g., an amount sufficient to modulate protein aggregation in a cell.

The term “therapeutically effective amount” is an amount that is effective to ameliorate a symptom of a disease. A therapeutically effective amount can, in some embodiments, be a “prophylactically effective amount” as prophylaxis can be considered therapy.

The term “administering” or “administration” of a drug and/or therapy to a subject (and grammatical equivalents of this phrase) refers to both direct or indirect administration, which may be administration to a subject by a medical professional, may be self-administration, and/or indirect administration, which may be the act of prescribing or inducing one to prescribe a drug and/or therapy to a subject.

The term “treating” or “treatment of” a disorder or disease refers to taking steps to alleviate the symptoms of the disorder or disease, or otherwise obtain some beneficial or desired results for a subject, including clinical results. Any beneficial or desired clinical results may include, but are not limited to, alleviation or amelioration of one or more symptoms of cancer or conditional survival and reduction of tumor load or tumor volume; diminishment of the extent of the disease; delay or slowing of the tumor progression or disease progression; amelioration, palliation, or stabilization of the tumor and/or the disease state; or other beneficial results.

The compounds of the present technology can exist as solvates, especially hydrates. Hydrates may form during manufacture of the compounds or compositions comprising the compounds, or hydrates may form over time due to the hygroscopic nature of the compounds. Compounds of the present technology can exist as organic solvates as well, including DMF, ether, and alcohol solvates among others. The identification and preparation of any particular solvate is within the skill of the ordinary artisan of synthetic organic or medicinal chemistry.

For the purposes of this specification and appended claims, unless otherwise indicated, all numbers expressing amounts, sizes, dimensions, proportions, shapes, formulations, parameters, percentages, parameters, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some aspects, ±100% in some aspects ±50%, in some aspects ±20%, in some aspects ±10%, in some aspects ±5%, in some aspects ±1%, in some aspects ±0.5%, and in some aspects ±0.1% from the specified amount, as such variations are appropriate to perform the disclosed methods or employ the disclosed compositions.

Unless defined otherwise, all technical and scientific terms used herein have the meaning commonly understood by a person skilled in the art to which this invention belongs.

Throughout this application, the text refers to various embodiments of the present compounds, compositions, and methods. The various embodiments described are meant to provide a variety of illustrative examples and should not be construed as descriptions of alternative species. Rather, it should be noted that the descriptions of various embodiments provided herein may be of overlapping scope. The embodiments discussed herein are merely illustrative and are not meant to limit the scope of the present technology.

### Formulations

Pharmaceutical Compositions

Herein disclosed are combinations of Bempedoic acid and Ezetimibe formulated in pharmaceutical compositions.

In one aspect, the present disclosure provides for a pharmaceutical composition comprising:

Bempedoic acid admixed with a lubricant selected from the group consisting of: colloidal silicon dioxide, sodium stearyl fumarate, and magnesium stearate;

Ezetimibe; and

a pharmaceutically acceptable excipient.

In some aspects, the present disclosure provides for a pharmaceutical composition wherein the composition comprises at least 40% and nor more than 95% Bempedoic acid by weight of the total composition and at least 0.5% and no more than 20% Ezetimibe by weight of the total composition.

In some aspects, the present disclosure provides for a pharmaceutical composition wherein the composition further comprises one or more of: magnesium stearate, hydroxypropyl cellulose (HPC-L), a pyrrolidone compound, a saccharide, an anionic surfactant, microcrystalline cellulose and a starch.

In some aspects, the present disclosure provides for a pharmaceutical composition wherein the composition further comprises each one of: magnesium stearate, hydroxypropyl cellulose (HPC-L), a pyrrolidone compound, a saccharide, an anionic surfactant, microcrystalline cellulose and a starch.

In some aspects, the present disclosure provides for a pharmaceutical composition wherein the microcrystalline cellulose, when present, comprises an average particle size of at least 100 μm and comprises a moisture content at least 3% and no more than 5% by weight of the microcrystalline cellulose.

In some aspects, the present disclosure provides for a pharmaceutical composition wherein the anionic surfactant, when present, is sodium lauryl sulfate.

In some aspects, the present disclosure provides for a pharmaceutical composition wherein the pyrrolidone compound, when present, is Povidone.

In some aspects, the present disclosure provides for a pharmaceutical composition wherein the saccharide, when present, is lactose monohydrate.

In some aspects, the present disclosure provides for a pharmaceutical composition wherein 1.03% by weight of the total magnesium stearate when present, has a particle size at least 45 μm and no more than 150 μm.

In some aspects, the present disclosure provides for a pharmaceutical composition wherein the composition is in the form of a tablet and further comprises a polyvinyl alcohol (PVA) based coating; and wherein the coating comprises: polyvinyl alcohol (PVA), glycerol monocaprylocaprate type 1, sodium lauryl sulfate, titanium dioxide and talc.

In some aspects, the present disclosure provides for a pharmaceutical composition wherein the amount of magnesium stearate is between 1 mg and 10 mg, the amount of hydroxypropyl cellulose (HPC-L) is between 5 mg and 25 mg, the amount of pyrrolidone compound is between 0.5 mg and 5 mg, the amount of saccharide is between 50 mg and 100 mg, the amount of anionic surfactant is between 0.5 mg and 5 mg, the amount of microcrystalline cellulose is between 25 mg and 100 mg and the amount of sodium starch glycolate is between 5 mg and 50 mg.

In some aspects, the present disclosure provides for a pharmaceutical composition wherein the amount of Bempedoic acid is between 80 mg and 250 mg; and the amount of Ezetimibe is between 5 mg and 25 mg. In some aspects, the amount of Bempedoic acid is between 100 mg and 200 mg; and the amount of Ezetimibe is between 7 mg and 15 mg. In some aspects, the amount of Bempedoic acid is between 150 mg and 200 mg; and the amount of Ezetimibe is between 9 mg and 12 mg.

In some aspects, the present disclosure provides for a pharmaceutical composition wherein the amount of Bempedoic acid is 180 mg and the amount of Ezetimibe is 10 mg.

In some aspects, the present disclosure provides for a pharmaceutical composition wherein the amount of Bempedoic acid is a fixed dose and the amount of Ezetimibe is a fixed dose.

In some aspects, the present disclosure provides for a pharmaceutical composition comprising Ezetimibe and Bempedoic acid as described herein that has improved flowability characteristics as described herein.

In some aspects, the present disclosure provides for a pharmaceutical composition comprising Ezetimibe and Bempedoic acid as described herein that has improved non-stickiness characteristics as described herein.

In some aspects, the present disclosure provides for a pharmaceutical composition comprising Ezetimibe and Bempedoic acid as described herein that has improved PK characteristics as described herein.

In some aspects, the present disclosure provides for a pharmaceutical composition comprising Ezetimibe and Bempedoic acid as described herein that has improved solubility characteristics as described herein.

In some aspects, the present disclosure provides for a pharmaceutical composition comprising Ezetimibe and Bempedoic acid as described herein that has improved extended release characteristics as described herein.

In some aspects, the present disclosure provides for a pharmaceutical composition comprising Ezetimibe and Bempedoic acid as described herein that has improved chemo-physical characteristics such as particle size, surface area, pore volume and other properties as described herein.

In some aspects, Ezetimibe in the pharmaceutical composition is amorphous. In some aspects, Ezetimibe in the pharmaceutical composition is a polymorph.

In some aspects, Bempedoic acid in the pharmaceutical composition is amorphous. In some aspects, Bempedoic acid in the pharmaceutical composition is a polymorph.

In some aspects, one of Bempedoic acid or Ezetimibe is amorphous. In some aspects, one of Bempedoic acid or Ezetimibe is a polymorph.

Formulations disclosed herein comprise the active compound(s), a pharmaceutically acceptable excipient, carrier, buffer, stabiliser or other materials well known to those skilled in the art. Such materials should be non-toxic and should not interfere with the efficacy of the active ingredient. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives can be included, as required.

The compounds in the combination, and solvates thereof, of the present disclosure can be formulated in pharmaceutical compositions. These compositions can comprise a pharmaceutically acceptable excipient, carrier, buffer, stabilizer or other materials well known to those skilled in the art. The precise nature of the carrier or other material can depend on the route of administration, e.g. oral, intravenous, cutaneous or subcutaneous, nasal, intramuscular, intraperitoneal routes.

Pharmaceutical compositions for oral administration can be in tablet, capsule, powder or liquid form. A tablet can include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol can be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives can be included, as required.

A composition can be administered alone or in combination with other treatments, either simultaneously or sequentially dependent upon the condition to be treated.

In general, the compounds of the present technology will be administered in a therapeutically effective amount by any of the accepted modes of administration for agents that serve similar utilities. The actual amount of the compound of the present technology, i.e., the active ingredient, will depend upon numerous factors such as the severity of the disease to be treated, the age and relative health of the subject, the potency of the compound used, the route and form of administration, and other factors well known to the skilled artisan. The drug can be administered at least once a day, preferably once or twice a day.

An effective amount of such agents can readily be determined by routine experimentation, as can the most effective and convenient route of administration and the most appropriate formulation. Various formulations and drug delivery systems are available in the art. See, e.g., Gennaro, A. R., ed. (1995) Remington's Pharmaceutical Sciences, 18th ed., Mack Publishing Co.

A therapeutically effective dose can be estimated initially using a variety of techniques well-known in the art. Initial doses used in animal studies may be based on effective concentrations established in cell culture assays. Dosage ranges appropriate for human subjects can be determined, for example, using data obtained from animal studies and cell culture assays.

An effective amount or a therapeutically effective amount or dose of an agent, e.g., a compound of the present technology, refers to that amount of the agent or compound that results in amelioration of symptoms or a prolongation of survival in a subject. Toxicity and therapeutic efficacy of such molecules can be determined by standard pharmaceutical procedures in cell cultures or experimental animals, e.g., by determining the LD₅₀ (the dose lethal to 50% of the population) and the ED₅₀ (the dose therapeutically effective in 50% of the population). The dose ratio of toxic to therapeutic effects is therapeutic index, which can be expressed as the ratio LD₅₀/ED₅₀. Agents that exhibit high therapeutic indices are preferred.

The effective amount or therapeutically effective amount is the amount of the compound or pharmaceutical composition that will elicit the biological or medical response of a tissue, system, animal or human that is being sought by the researcher, veterinarian, medical doctor or other clinician. Dosages particularly fall within a range of circulating concentrations that includes the ED₅₀ with little or no toxicity. Dosages may vary within this range depending upon the dosage form employed and/or the route of administration utilized. The exact formulation, route of administration, dosage, and dosage interval should be chosen according to methods known in the art, in view of the specifics of a subject's condition.

Dosage amount and interval may be adjusted individually to provide plasma levels of the active moiety that are sufficient to achieve the desired effects; i.e., the minimal effective concentration (MEC). The MEC will vary for each compound but can be estimated from, for example, in vitro data and animal experiments. Dosages necessary to achieve the MEC will depend on individual characteristics and route of administration. In cases of local administration or selective uptake, the effective local concentration of the drug may not be related to plasma concentration.

The amount of agent or composition administered may be dependent on a variety of factors, including the sex, age, and weight of the subject being treated, the severity of the affliction, the manner of administration, and the judgment of the prescribing physician.

The present technology is not limited to any particular composition or pharmaceutical carrier, as such may vary. In general, compounds of the present technology will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of the present technology is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes therapeutic agents (which are formulated in a liquid form) to spray as a mist that is carried into the subject's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the subject's inspiratory air-stream during breathing by the device. In order to achieve a free flowing powder, therapeutic agent is formulated with an excipient such as lactose. A measured amount of therapeutic agent is stored in a capsule form and is dispensed with each actuation.

Pharmaceutical dosage forms of a compound of the present technology may be manufactured by any of the methods well-known in the art, such as, for example, by conventional mixing, sieving, dissolving, melting, granulating, dragee-making, tabletting, suspending, extruding, spray-drying, levigating, emulsifying, (nano/micro-) encapsulating, entrapping, or lyophilization processes. As noted above, the compositions of the present technology can include one or more physiologically acceptable inactive ingredients that facilitate processing of active molecules into preparations for pharmaceutical use.

Tablets

In some aspects, the present disclosure provides for a monolayer or a bilayer tablet as described herein. The monolayer or a bilayer tablet comprise a composition of Bempedoic acid and Ezetimibe, and optionally one or more pharmaceutically acceptable excipients as described herein.

In some aspects, the present disclosure provides for a bilayer tablet comprising Bempedoic acid and Ezetimibe, wherein the first layer comprises:

Ezetimibe granulated with a pharmaceutically acceptable excipient;

and wherein the second layer comprises:

Bempedoic acid granulated with a lubricant and a pharmaceutically acceptable excipient, wherein the lubricant is selected from the group consisting of: colloidal silicon dioxide, sodium stearyl fumarate, and magnesium stearate.

In some aspects, the present disclosure provides for a bilayer tablet wherein the Bempedoic acid is at least 20% and no more than 64% by weight of the total tablet and Ezetimibe is at least 1% and no more than 7% by weight of the total tablet.

In some aspects, the present disclosure provides for a bilayer tablet wherein the first layer is at least 0.1% and no more than 23% by weight of the total tablet and the second layer is at least 0.1% and no more than 74% by weight of the total tablet.

In some aspects, the present disclosure provides for a bilayer tablet wherein the Friability of the tablet is at least 0.01% and no more than 0.1%. Friability is a routine test for tablet compositions, the skilled artisan can determine Friability by a number of methods. For example, the skilled artisan may determine Friability of compositions of the present disclosure by the methods described in the monograph USP Tablet Friability <1216>, which describes the recommended apparatus and the test procedure. Herein, USP Tablet Friability <1216> is incorporated by reference in its entirety.

In some aspects, the present disclosure provides for a monolayer or a bilayer tablet comprising Ezetimibe and Bempedoic acid as described herein that has improved physical characteristics such as Friability and other properties as described herein.

Granulated Compositions

In some aspects, the present disclosure provides for a granulated composition comprising: Bempedoic acid admixed with a lubricant selected from the group consisting of: colloidal silicon dioxide, sodium stearyl fumarate, and magnesium stearate.

In some aspects, the present disclosure provides for a granulated composition wherein the composition further comprises a pharmaceutically acceptable excipient.

In some aspects, the present disclosure provides for a granulated composition wherein the lubricant is colloidal silicon dioxide.

In some aspects, the present disclosure provides for a granulated composition wherein the composition has a bulk density of at least 0.25 gm/ml and no more than 0.55 gm/ml.

In some aspects, the present disclosure provides for a granulated composition wherein the composition has a Carr's Index of at least 10 and no more than 30. The Carr index relates to the compressibility and hence, the flowability of a material. Carr's index is a routine measurement for one skilled in the art and numerous methods may be employed. For example, the skilled artisan can use the methods, apparatus and procedures described in the monograph USP29-NF24 (page 2638) to determine the Carr's Index of a composition of the present disclosure. Herein, the entirety of monograph USP29 is incorporated by reference.

In some aspects, the present disclosure provides for a granulated composition wherein the granules of the composition have an angle of repose of at least 20 no more than 45. The morphology of a given material and its composition both affect the angle of repose. The angle of repose is related to the density, surface area, shapes of the particles, and the coefficient of friction of the material. One skilled in the art can use numerous methods to determine the angle of repose, one example would be to use the methods and procedures described in USP29.

In some aspects, the present disclosure provides for a granulated composition wherein the Bempedoic acid is present in an amount of at least 50% and no more than 95% by weight of the total formulation.

In some aspects, the present disclosure provides for a granulated composition wherein the composition further comprises hydroxypropyl cellulose (HPC-L). In some aspects, the present disclosure provides for a granulated composition wherein the composition further comprises microcrystalline cellulose. In some aspects, the amount of the HPC-L is at least 3% and no more than 10% by weight of the total formulation; the amount of the Bempedoic acid is at least 50% and no more than 95% by weight of the total formulation; and the amount of the microcrystalline cellulose is at least 1% and no more than 20% by weight of the total formulation.

In some aspects, Bempedoic acid in the granulated composition is amorphous. In some aspects, Bempedoic acid in the granulated composition is a polymorph.

### Kits

The present disclosure also provides for a kit comprising one or more compositions which itself comprises Bempedoic acid or a combination of Bempedoic acid and Ezetimibe, and instructions for use. The present disclosure further provides for a kit comprising one or more compositions.

The present disclosure further provides for a kit comprising one or more compositions comprising Bempedoic acid or Bempedoic acid and Ezetimibe, and optionally the composition and/or kit includes at least one pharmaceutically acceptable carrier or excipient.

In some aspects, the disclosure provides for a kit comprising a combination composition comprising: Bempedoic acid admixed with a lubricant selected from the group consisting of: colloidal silicon dioxide, sodium stearyl fumarate, and magnesium stearate, and Ezetimibe, and optionally at least one pharmaceutically acceptable carrier or excipient.

In one aspect, the present disclosure provides for a kit and instructions for use, where the instructions for use recite a process or recite directions for mixing the one or more of the granulated compositions or one or more pharmaceutical compositions or one or more tablets with one or more compositions.

Individual components of the kit can be packaged in separate containers and, associated with such containers, can be a notice in the form prescribed by a governmental agency regulating the manufacture, use or sale of pharmaceuticals or biological products, which notice reflects approval by the agency of manufacture, use or sale. The kit may optionally contain instructions or directions outlining the method of use or administration regimen for the antigen-binding construct.

When one or more components of the kit are provided as solutions, for example an aqueous solution, or a sterile aqueous solution, the container means may itself be an inhalant, syringe, pipette, eye dropper, or other such like apparatus, from which the solution may be administered to a subject or applied to and mixed with the other components of the kit.

The components of the kit may also be provided in dried or lyophilized form and the kit can additionally contain a suitable solvent for reconstitution of the lyophilized components. Irrespective of the number or type of containers, the kits described herein also may comprise an instrument for assisting with the administration of the composition to a patient. Such an instrument may be an inhalant, nasal spray device, syringe, pipette, forceps, measured spoon, eye dropper or similar medically approved delivery vehicle.

### Syntheses of Bempedoic Acid (ETC-1002) and Ezetimibe

The structure of ETC-1002 is:

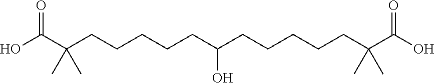 [SMILES: CC(C)(CCCCCC(O)CCCCCC(C)(C)C(=O)O)C(=O)O]

ETC-1002 and the processes for the synthesis of ETC-1002 are disclosed in issued U.S. Pat. No. 7,335,799. The details of this process can be found in the published U.S. Patent Publication No. US2005/0043278A1, in paragraphs [0247]-[0343] of the specification, which is herein incorporated by reference.

The structure of Ezetimibe is:

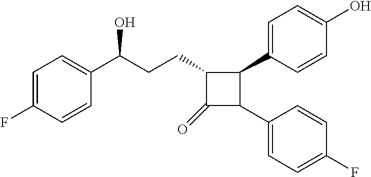 [SMILES: O=C1C(c2ccc(F)cc2)[C@H](c2ccc(O)cc2)[C@H]1CC[C@H](O)c1ccc(F)cc1]

Ezetimibe and the synthesis of Ezetimibe is also known. Ezetimibe and the process for the synthesis of Ezetimibe is disclosed in the issued U.S. Pat. No. 5,631,365. The details of this process can be found in the specification, beginning on page 4 right column, line 43 through page 11 right column, line 65, each of which is herein incorporated by reference.

Additionally, both compounds are small molecules under 500 Da in weight and the skilled artisan will be able to use synthetic reference texts to synthesize the desired final compound from readily available or commercially available chemicals. Such references include, but are not limited to: as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5^{th} Edition, 2001), and Larock's Comprehensive Organic Transformations (VCH Publishers Inc., 1989), T. W. Greene and P. G. M. Wuts, Protecting Groups in Organic Synthesis, Third Edition, Wiley, New York, 1999.

### Processes

Granulating

In some aspects, the present disclosure provides for a process of granulating Bempedoic acid, the process comprising:

dry mixing: Bempedoic acid, a lubricant and a pharmaceutically acceptable excipient, wherein the lubricant is selected from the group consisting of: colloidal silicon dioxide, sodium stearyl fumarate, and magnesium stearate;

separately mixing an aqueous preparation of HPC-L;

blending the dry mixture and aqueous preparation; and

granulating the blend.

In some aspects, the present disclosure provides for a process of granulating Bempedoic acid, wherein the process further comprises using a rapid mixer to granulate the blend.

In some aspects, the present disclosure provides for a process of granulating Bempedoic acid, wherein the process further comprises drying the blend.

In some aspects, the present disclosure provides for a process of granulating Bempedoic acid, wherein the process further comprises milling and co-sifting the blend.

In some aspects, the present disclosure provides for a process of granulating Bempedoic acid, wherein the dry mixing is carried out for at least forty-five (45) minutes.

In some aspects, the present disclosure provides for a process of granulating Bempedoic acid, wherein the dry mixing is carried out for no more than twenty four (24) hours in duration.

Manufacturing

In some aspects, the present disclosure provides for a process of manufacturing a monolayer tablet comprising Ezetimibe and Bempedoic acid, the process comprising:

granulating a composition comprising Ezetimibe and granulating a composition comprising Bempedoic acid, where each composition is granulated separately;

blending the Ezetimibe and Bempedoic acid granulations together;

compressing the blend into a single layer; and

coating the single layer.

In some aspects, the present disclosure provides for a process of manufacturing a monolayer tablet wherein the process further comprises drying the tablets.

In some aspects, the present disclosure provides for a process of manufacturing a monolayer tablet wherein the coating comprises one or more of: PVA, glycerol monocaprylocaprate type 1, sodium lauryl sulfate, titanium dioxide and talc.

In some aspects, the present disclosure provides for a process of manufacturing a monolayer tablet wherein the coating comprises each one of: PVA, glycerol monocaprylocaprate type 1, sodium lauryl sulfate, titanium dioxide and talc.

In some aspects, the present disclosure provides for a process of manufacturing a bilayer tablet comprising Ezetimibe and Bempedoic acid, the process comprising:

granulating a composition comprising Ezetimibe and granulating a composition comprising Bempedoic acid, where each composition is granulated separately;

blending the Ezetimibe granules with a pharmaceutically acceptable excipient;

blending the Bempedoic acid granules with a pharmaceutically acceptable excipient;

compressing the Ezetimibe and Bempedoic acid blends into a single composition containing two (2) separate layers; and

coating the composition.

In some aspects, the present disclosure provides for a process of manufacturing a bilayer tablet wherein the process further comprises drying the tablets.

In some aspects, the present disclosure provides for a process of manufacturing a bilayer tablet wherein the Bempedoic acid composition includes colloidal silicon dioxide, sodium stearyl fumarate, or magnesium stearate.

In some aspects, the present disclosure provides for a process of manufacturing a bilayer tablet wherein the Ezetimibe composition includes an anionic surfactant.

### Methods of Use

In one aspect, the present disclosure provides for methods for the treatment or prevention of a cardiovascular disease, said methods comprising administering a pharmaceutical composition comprising: Ezetimibe and Bempedoic acid admixed with a lubricant selected from the group consisting of: colloidal silicon dioxide, sodium stearyl fumarate, and magnesium stearate.

In one aspect, the present disclosure provides for methods for the treatment or prevention of a cardiovascular disease, said methods comprising administering a pharmaceutical composition comprising:

Bempedoic acid admixed with a lubricant selected from the group consisting of: colloidal silicon dioxide, sodium stearyl fumarate, and magnesium stearate;

Ezetimibe; and

a pharmaceutically acceptable excipient to a subject in need thereof.

In one aspect, the present disclosure provides for methods for the treatment or prevention of a cardiovascular disease, said methods comprising administering a pharmaceutical composition comprising:

a fixed dose of Bempedoic acid admixed with a lubricant selected from the group consisting of: colloidal silicon dioxide, sodium stearyl fumarate, and magnesium stearate;

a fixed dose of Ezetimibe; and

a pharmaceutically acceptable excipient to a subject in need thereof.

In some aspects, the present disclosure provides for methods for the treatment or prevention of a cardiovascular disease, said methods comprising administering a fixed dose of a pharmaceutical composition disclosed herein.

In some aspects, the present disclosure provides for methods for the treatment or prevention of a dyslipidemia, said methods comprising administering a pharmaceutical composition comprising: Ezetimibe and Bempedoic acid admixed with a lubricant selected from the group consisting of: colloidal silicon dioxide, sodium stearyl fumarate, and magnesium stearate.

In some aspects, the present disclosure provides for methods for the treatment or prevention of a dyslipidemia, said methods comprising administering a pharmaceutical composition comprising:

Bempedoic acid admixed with a lubricant selected from the group consisting of: colloidal silicon dioxide, sodium stearyl fumarate, and magnesium stearate;

Ezetimibe; and

a pharmaceutically acceptable excipient to a subject in need thereof.

Dyslipidemias which the compositions of the present invention are useful for preventing or treating include but are not limited to hyperlipidemia and low blood levels of high density lipoprotein (HDL) cholesterol. In certain embodiments, the hyperlipidemia for prevention or treatment by the compounds of the present invention is familial hypercholesterolemia; familial combined hyperlipidemia; reduced or deficient lipoprotein lipase levels or activity, including reductions or deficiencies resulting from lipoprotein lipase mutations; hypertriglyceridemia; hypercholesterolemia; high blood levels of urea bodies (e.g. .beta.-OH butyric acid); high blood levels of Lp(a) cholesterol; high blood levels of low density lipoprotein (LDL) cholesterol; high blood levels of very low density lipoprotein (VLDL) cholesterol and high blood levels of non-esterified fatty acids.

The present disclosure provides for methods for altering lipid metabolism in a patient, e.g., reducing LDL in the blood of a patient, reducing free triglycerides in the blood of a patient, increasing the level of high density lipoprotein (HDL) in the blood of a patient, reducing the level of very low density lipoprotein (VLDL) in the blood of a patient, reducing the number of very low density lipoprotein (VLDL) particles in the blood of a patient, reducing the size of VLDL particles in the blood of a patient, increasing the level of apolipoprotein A-1 (ApoA1) in the blood of a patient, reducing the he ratio of apolipoprotein B (ApoB) to apolipoprotein A-1 (ApoA1) in the blood of a patient, increasing the ratio of HDL to LDL in the blood of a patient, and inhibiting saponified and/or non-saponified fatty acid synthesis, said methods comprising administering to the patient a administering a pharmaceutical composition comprising: Bempedoic acid admixed with a lubricant selected from the group consisting of: colloidal silicon dioxide, sodium stearyl fumarate, and magnesium stearate; Ezetimibe; and a pharmaceutically acceptable excipient in an amount effective to alter lipid metabolism.

### EXAMPLES

Formulation development was initiated with the characterization of both APIs along with an evaluation of individual reference products. Dissolution profile, and drug-drug and drug-excipient compatibility were characterized. Additionally, the process operations were defined and a process control strategy was examined. Manufacturing process developments identified wet granulation, blending, compression, and coating as the main process options. Risk was assessed throughout development to identify high risk formulations and process variables and to determine a path forward to develop a control strategy. Risk assessments were updated after development to capture the reduction of risk due to improved product and process understanding.

Dissolution parameters (e.g. dissolution media, volume, apparatus, and agitation speed) were selected based on the pending USP monograph for Ezetimibe and the analytical methods listed in IND #106,654 for Bempedoic acid. The particle size of Ezetimibe is critical for dissolution; hence the micronized form of each API was used. Limits of single known and unspecified impurities were set in accordance with ICH Q3B (R2) to control impurity in finished drug products.

The quality target product profile (QTPP) is defined based on properties of the drug substance, characterization of the reference product, and other information available for the two compounds. Critical quality attributes (CQAs) were identified as those drug product attributes that could impact the risk to a patient (safety and efficacy). The present disclosure focuses on those CQAs that are impacted to affect a realistic change to the drug product formulation or manufacturing process, namely: assay, content uniformity, dissolution, and degradation products.

Initially, it was found that Bempedoic acid exhibits sticky behavior as a granulated material. This behavior leads to extraordinary challenges to making tablets by compression. To address this challenge, the granulation process was modified by treating the active with a mixture of colloidal silicon and a cellulosic binder.

Granulation of bempedoic acid is altered with a mixture of colloidal silicon dioxide and hydroxypropyl cellulose (HPC-L). This blend was further granulated using a solution of HPC-L. The solubility of Ezetimibe was improved by homogenization with sodium lauryl sulfate (SLS) and Povidone. This dispersion was then used to granulate excipient blend using top spray attachment of fluid bed processor. Opadry AMBII white 88A180040 was selected as the coating agent to avoid the incompatibility of ezetimibe with polyethylene glycol and/or polyvinyl alcohol.

For the purposes of this development, drug products containing bempedoic acid film coated tablet (180 mg) from Esperion Therapeutics, Inc. and Zetia® (Ezetimibe, 10 mg) manufactured by MSD International GmbH (NDA No: 21445) were utilized as the reference products.

### 1 Development Objectives

The objective was broken down into the following activities:
Study the compatibility of both drug substances together and with selected excipients.
Develop a product with a satisfactory process and stability profile.
Match the release profile of FDC product to individual reference product in QC and discriminatory dissolution media.
Study the pharmacokinetics of the developed FDC product along with the reference product (co-administered ETC-1002 (180 mg) tablets and Zetia (10 mg).

### 2 Reference Listed Drug (RLD) Product Descriptions

### Zetia® (Ezetimibe)

Zetia is commercially available as a tablet for oral administration containing 10 mg of Ezetimibe. It was approved in the United States in 2002 (NDA No: 21445).

### 3.1.2 Bempedoic Acid (ETC-1002) Tablet:

Bempedoic acid (ETC-1002) is an innovative, first-in-class, small molecule designed to lower elevated levels of LDL-C and to avoid side effects associated with existing LDL-C lowering therapies. It is intended to be taken once a day.

### Example 1

### 3 Drug Release

Both ezetimibe and Bempedoic acid are BCS Class-II compounds (poorly soluble and highly permeable) and therefore drug release is a rate limiting process for absorption. A thorough evaluation of the drug release profile of the reference products was carried out.

### 3.2.1 Multimedia Dissolution of Zetia:

Dissolution characterization was performed in accordance with pending USP monograph (500 mL 0.45% SLS in 0.05 M acetate buffer, pH 4.5 dissolution media, Apparatus-II, 50 rpm). The temperature of the dissolution media was maintained at 37±0.5° C. and the released drug concentration was determined using HPLC. The drug release was also studied in two additional media at alternate pH (0.1 N HCl with 0.45% w/v SLS and pH 6.8 phosphate buffer with 0.45% w/v SLS). Multimedia dissolution profiles are shown in Table 1 and FIG.

TABLE 1
Multimedia Dissolution Data for Zetia

| | Product | | |
|---|---|---|---|
| | Zetia | | |
| | Media | | |
| | | | 0.45% SLS in |
| | | 0.45% SLS in | 0.05M Na |
| | 0.45% SLS | 0.05M Acetate | Phosphate |
| | in 0.1N HCl | Buffer pH 4.5 | Buffer pH 6.8 |
| Time (min) | % Drug release | | |
| 10 | 73.6 | 88.4 | 86.5 |
| 20 | 89.5 | 93.2 | 99.1 |
| 30 | 91.2 | 93.7 | 99.8 |
| 45 | 92.3 | 93.5 | 102.7 |

Zetia exhibited rapid dissolution with more than 80% drug release within 15 minutes in all three studied media. The aqueous solubility of ezetimibe is very low; however the use of 0.45% SLS enables sink condition.

### 3.2.2 Multimedia Dissolution of Bempedoic Acid Tablets

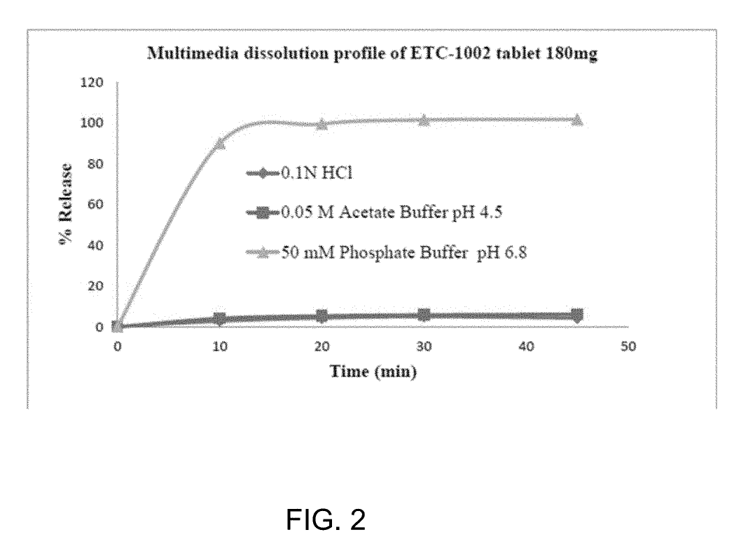

Dissolution characterization was performed in accordance with the dissolution method detailed in IND #106,654. The dissolution conditions were 900 mL phosphate buffer, pH 6.8, using Apparatus-II at 50 rpm. The data are presented in Table 2 and FIG. 2.

TABLE 2
Multimedia dissolution data for Bempedoic Acid tablets

| | Product | | |
|---|---|---|---|
| | Bempedoic Acid Tablets | | |
| | Media | | |
| | | Acetate Buffer | Phosphate Buffer |
| | 0.1N HCl | pH 4.5 | pH 6.8 |
| Time (min) | % Drug release | | |
| 10 | 2.9 | 4.2 | 90.2 |
| 20 | 4.4 | 5.4 | 99.6 |
| 30 | 5.1 | 5.9 | 101.6 |
| 45 | 4.4 | 6.2 | 101.8 |

Bempedoic acid has two carboxylic acidic functional groups (—COOH) which make solubility pH dependent, as observed in this multimedia dissolution study. Low dissolution values were observed in 0.1 N HCl and acetate buffer, pH 4.5, however in the QC medium (phosphate buffer, pH 6.8) more than 85% of the drug was released in 20 minutes.

### 3.3 Physicochemical Characterization

The physicochemical characterizations of the reference products are summarized in Table 3.

TABLE 3
Physical Attributes of Reference Products

| | | | Bempedoic |
|---|---|---|---|
| Parameter | Zetia | | Acid Tablets |
| Brand Name | Zetia ® (ezetimibe) | | Not Applicable |
| Generic Name | Ezetimibe 10 mg | | Bempedoic acid |
| | | | 180 mg |
| Manufacturer | MSD International | | Esperion |
| | GmbH, Inc., a subsidiary | | Therapeutic. Inc. |
| | of Merck & Co. Inc. | | |
| Dosage form | Tablet | | Tablet |
| Label claim | Each uncoated tablet | | Each film coated |
| | contain 10 mg ezetimibe | | tablet contain 180 mg |
| | | | bempedoic acid |
| Batch No. | L015901 | | N460335 |
| Expiry date | December 2017 | | Not available |
| Pack | HDPE (NDC | | HDPE container |
| | 66582-414-31) | | Pack of 30 tablets |
| | Pack of 30 tablets | | |
| Appearance | White elongated beveled | | Oval, white to off |
| | tablet debossed “414” on | | white film coated |
| | one side and plain on | | tablet debossed with |
| | other side | | “ABC” on one |
| | | | face and debossed |
| | | | with 000 on the |
| | | | opposite face |
| Coated/Uncoated | Uncoated | | Coated |
| Average weight (mg) | 100.2 | | 310.3 |
| Length (mm) | 8.24-8.26 | | 14.03-14.06 |
| Thickness (mm) | 4.65-4.67 | | 4.62-4.63 |
| Hardness (N) | 48-50 | | 125-135 |
| Disintegration Time | 3-4 min | | 5-6 min |
| pH (1 tablet in 100 mL | 6.8 | | 6.3 |
| of Purified water) | | | |
| Water Content by Karl | 2.46 | | 5.48 |
| Fischer (% w/v) | | | |
| Assay (%) | 100.0 | | 99.8 |
| Related Substance | Impurity | % | No Unspecified |
| | | | impurities detected |
| | Cyclic ether | 0.07 | |
| | RRS isomer | Not detected | |
| | EZT⁽¹⁾ 21 | BLOQ | |
| | (Keto | | |
| | impurity) | | |
| | Single | 0.06 | |
| | maximum | | |
| | unspecified | | |
| | impurities | | |
| | Total | 0.13 | |
| | impurities | | |

| | | |
|---|---|---|
| Excipients | Croscarmellose sodium, | Sodium starch |
| | lactose monohydrate, | glycolate, lactose |
| | magnesium stearate, | monohydrate, |
| | microcrystalline cellulose, | microcrystalline |
| | povidone and sodium | cellulose, |
| | lauryl sulfate | hydroxypropyl |
| | | cellulose, colloidal |
| | | silicon dioxide, |
| | | magnesium stearate, |
| | | Opadry white- |
| | | 85F18422 |

⁽¹⁾ EZT (ezetimibe)

### 3.4 Composition:

Based on the reference product labeling and relevant literature, Table 4 lists the predicted composition of Zetia (10 mg). The composition of bempedoic acid 180 mg tablet, provided by Esperion, is detailed in Table 5.

TABLE 4
Predicted Composition of Zetia 10 mg Tablet

| Sr. No. | Ingredients | mg/tablet |
|---|---|---|
| 01 | Ezetimibe | 10 |
| 02 | Lactose monohydrate | 50-60 |
| 03 | Microcrystalline cellulose | 10-30 |
| 04 | Povidone | 3-5 |
| 05 | Croscarmellose sodium | 6-10 |
| 06 | Sodium lauryl sulfate | 1-3 |
| 07 | Magnesium stearate | 1 |

| | |
|---|---|
| Total weight of Zetia | 100 |

TABLE 5
Composition of Bempedoic Acid 180 mg Tablet

| Sr. No. | Ingredients | mg/tablet |
|---|---|---|
| 01 | Bempedoic acid | 180 |
| 02 | Microcrystalline cellulose | 51 |
| 03 | Lactose | 30 |
| 04 | Sodium starch glycolate | 21 |
| 05 | Hydroxypropyl cellulose | 12 |
| 06 | Colloidal silicon dioxide | 1.5 |
| 07 | Magnesium stearate | 4.5 |
| 08 | Opadry white (85F18422) | 9 |

| | |
|---|---|
| Total weight of film coated bempedoic acid tablet | 309 |

### 4. Fixed Dose Combination (FDC) Product

### 4.1 Quality Target Product Profile (QTPP)

The quality target product profile (QTPP) is a prospective summary of the characteristics of a drug product that ideally will be achieved to ensure the desired quality, taking into account safety and efficacy of the drug product. The QTPP is an essential element of a Quality by Design (QbD) approach and forms the basis of design of the drug product. The QTPP set for the FDC product is as listed in Table 6.

TABLE 6
Quality Target Product Profile for FDC

| QTPP Element | Target | Justification |
|---|---|---|
| Dosage form | Tablet | Tablet is the most preferred and convenient |
| | | dosage form for administration to patient. |
| Route of | Oral | It is the most preferred route of |
| administration | | administration to achieve desired |
| | | efficacy and safety targets. |
| Dosage strength | 10 mg ezetimibe + 180 | Fixed dose combination for effective |
| | mg Bempedoic acid | lowering of lipid levels. |
| Pharmacokinetics | Bioequivalent to | Bioequivalence requirement to both |
| | individual reference | individual reference products. Need to |
| | drug product RLD | ensure rapid onset of action and |
| | | efficiency. |
| Stability | At least 24-month shelf- | Needed for commercialization. |
| | life at room temperature | Equivalent to or better than reference |
| | | product. |
| Drug product | Physical attributes | Pharmaceutical equivalence requirement. |
| quality | Identification | Meeting the same compendia or other |
| attributes | Assay | applicable (quality) standards (i.e., |
| | Content uniformity | identity, assay, purity, and quality) |
| | Dissolution | |
| | Degradation products | |
| | Residual solvents | |
| | Water content | |
| | Microbial limits | |
| Container | HDPE bottles with Child | Needed to achieve the target shelf-life and to |
| closure system | Resistant (CR) Caps with | ensure integrity during shipping for safety |
| | desiccant | and commercial requirements. |
| Administration/ | To be determined based on | For effectiveness of the proposed dosage |
| concurrence with | outcome of the PK studies | form. |
| labeling | | |

Table 7 summarizes the quality attributes of ezetimibe (10 mg) and bempedoic acid (180 mg) FDC and indicates which attributes were classified as critical quality attributes (CQAs). The CQAs that have the potential to be impacted by the formulation and/or process variables were investigated during development studies. CQAs which are unlikely to be impacted by formulation and/or process variables were not be studied. However, these CQAs are still target elements of the QTPP and are ensured through a good pharmaceutical quality system and the control strategy.

TABLE 7
FDC product CQAs

| Quality Attributes of the | | | Is this a | |
|---|---|---|---|---|
| Drug Product | | Target | CQA? | Justification |
| Physical | Appearance | Color and shape | No | Color, shape and appearance are not |
| Attributes | | acceptable to the | | directly linked to safety and efficacy. |
| | | patient. No | | Therefore, they are not critical. The |
| | | visual tablet | | target is set to ensure patient |
| | | defects observed | | acceptability. |
| | Odor | No unpleasant | No | In general, a noticeable odor is not |
| | | odor | | directly linked to safety and efficacy, |
| | | | | but odor can affect patient |
| | | | | acceptability. For this product, neither |
| | | | | the drug substance nor the excipients |
| | | | | have an unpleasant odor. |
| | Size | Similar or | No | For ease of swallowing and patient |
| | | acceptably | | acceptance. |
| | | greater than | | |
| | | current product | | |
| | Friability | NMT 1.0% w/w | No | Friability is a routine test per |
| | | | | compendia requirements for tablets. A |
| | | | | target of NMT 1.0% w/w of mean |
| | | | | weight loss assures a low impact on |
| | | | | patient safety and efficacy and |
| | | | | minimizes customer complaints. |

| | | | |
|---|---|---|---|
| Identification | Positive for | Yes | Identification is critical to safety and |
| | ezetimibe and | | efficacy. This CQA can be effectively |
| | bempedoic acid | | controlled by the quality management |
| | | | system and will be monitored at drug |
| | | | product release. This CQA will not be |
| | | | studied. |
| Assay | 100% w/w of | Yes | Assay variability can affect safety and |
| | label claim of | | efficacy. Process variables may affect |
| | both actives | | the assay of the drug product. Hence, |
| | | | assay will be evaluated throughout the |
| | | | development. |
| Content | Conforms to | Yes | Variability in content uniformity will |
| Uniformity (CU) | USP <905> | | affect safety and efficacy. Both |
| | Uniformity of | | formulation and process variables may |
| | Dosage Units | | impact content uniformity. Hence, this |
| | | | CQA will be evaluated. |
| Dissolution | NLT 80% (Q) at | Yes | Failure to meet the dissolution |
| | 30 minutes for | | specification can impact bioavailability. |
| | both ezetimibe | | Formulation and process variables both |
| | and bempedoic | | affect the dissolution profile. This CQA |
| | acid | | will be investigated throughout |
| | | | development |
| Degradation Products | Specified | Yes | Impurities will be monitored based on |
| | impurities: | | ICH identification and qualification |
| | NMT 0.2% | | threshold. This CQA will be |
| | Any | | investigated throughout formulation |
| | unidentified | | development and stability evaluation. |
| | impurity: NMT | | |
| | 0.2% | | |
| | Total impurities: | | |
| | NMT 1.0% | | |
| Residual Solvents | USP <467> | Yes | Residual solvents can impact safety. |
| | option 1 | | However, no solvent is used in the drug |
| | | | product manufacturing process and the |
| | | | drug product complies with |
| | | | <467>option 1.Therefore, formulation |
| | | | and process variables are unlikely to |
| | | | impact this CQA. |
| Microbial | Complies with | Yes | Non-compliance with microbial limits |
| enumeration test | USP <61> and | | will impact patient safety. It will be |
| | USP <62> | | controlled during the manufacturing |
| | | | process. Therefore, this CQA will not |
| | | | be discussed in detail. |

### 4.2 Dissolution Method

### 4.2.1 Dissolution Method Development for Bempedoic Acid and Ezetimibe FDC Product:

Table 8 shows the dissolution method used to measure drug release from the FDC product. Drug release profiles for both actives were estimated using common chromatographic conditions with different injection volumes.

TABLE 8
Dissolution Method for QC Testing
of Individual Reference Products

| | Ezetimibe Tablets | Bempedoic Acid Tablets |
|---|---|---|
| | (Per USFDA data base) | Information |
| Apparatus | USP Apparatus-II | USP Apparatus-II |
| Rotation speed | 50 | 50 |
| (rpm) | | |
| Volume (mL) | 500 | 900 |
| Dissolution | 10, 20, 30 and | 10, 20, 30, 45 min, |
| time | 45 minutes | and infinity |
| | | time point (additional |
| | | 15 min at 250 RPM) |
| Dissolution | in 0.05M acetate | 50 mM phosphate |
| media | buffer, pH 4.5 | buffer, pH 6.8 |

### 4.2.2 Method Development of Ezetimibe Discriminatory Dissolution Method

Ezetimibe exhibits poor aqueous solubility (insoluble in aqueous media at all pH); hence incorporation of SLS in the dissolution media is necessary.

QC dissolution medium, 0.45% SLS in 0.05 M acetate buffer, pH 4.5 yields more than 85% of drug release within 15 minutes. Reduced concentrations of SLS and variable volumes of dissolution media were evaluated to identify suitable discrimination for dissolution of reference product.

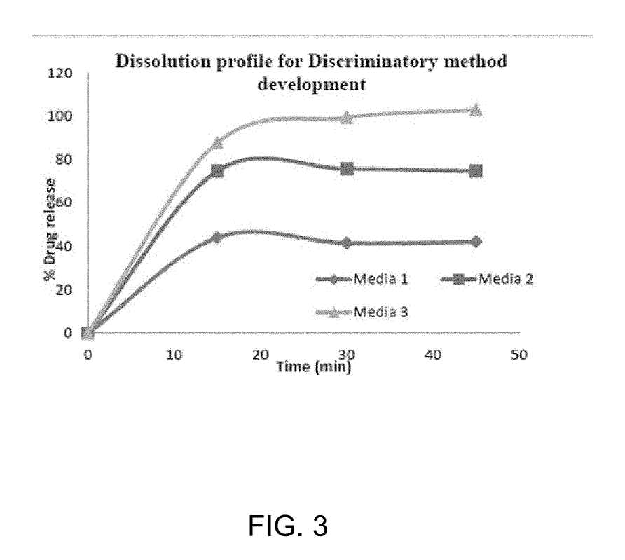

Table 9 and FIG. 3 show the dissolution data for reference product combinations at various dissolution conditions.

TABLE 9
Dissolution Profile of the Combined Reference Products
with Different Dissolution Media

| | Product | | |
|---|---|---|---|
| | Bempedoic Acid + Zetia Reference Product | | |
| | Media | | |
| | Medium 1: 500 | Medium 2: | Medium 3: |
| | mL; 0.1% | 900 mL; | 900 mL; |
| | SLS in | 0.1% SLS | 0.2% SLS |
| | acetate buffer, | in acetate | in acetate |
| | pH 4.5 | buffer, pH 4.5 | buffer pH 4.5 |
| Time (min) | % Drug release (ezetimibe) | | |
| 15 | 44.0 | 74.7 | 87.8 |
| 30 | 41.5 | 75.6 | 99.3 |
| 45 | 4.20 | 74.6 | 102.9 |

Based on the obtained dissolution profiles, Medium 2 (900 mL acetate buffer, pH 4.5 with 0.1% SLS) was proposed to be discriminatory medium. Media 1 and 3 were not selected because Medium 3 (0.2% SLS) shows dose dumping (as was also observed in the QC release media) and Medium 1 (reduced volume of media) showed inadequate sink conditions to release complete drug.

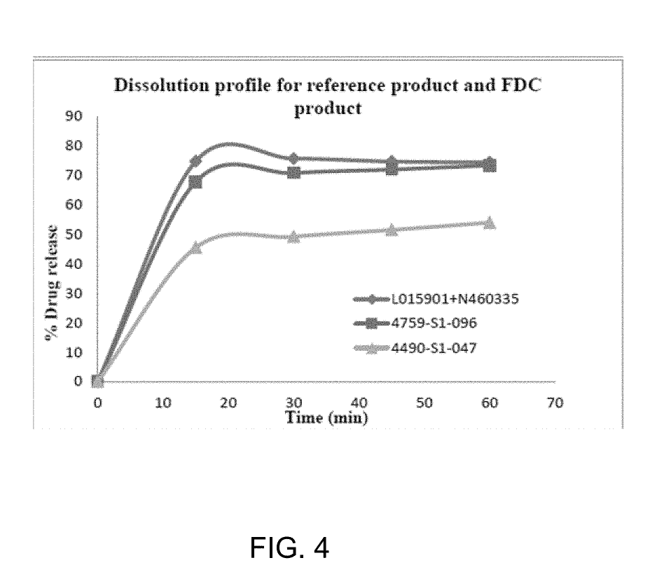

The FDC prototype formulation (Batch no: 4759-S1-096) shows comparable release to the combined reference product (Zetia (10 mg)+bempedoic acid tablet (180 mg)) in discriminatory dissolution media and the discriminatory power of the method is demonstrated in the example shown in Table 10 and FIG. 4.

To understand the discriminatory power of the dissolution media, the dissolution profiles of the following two batches was monitored:

Batch No: 4759-S1-096—Homogenized ezetimibe with top spray granulation
Batch No: 4490-S1-047—Ezetimibe mixed with excipients (diluents & super-disintegrant) followed by wet granulation.

The batch with homogenized API, Batch No: 4759-S1-096, was expected to have a faster release profile compared to the other batch where ezetimibe was not homogenized.

TABLE 10
Evaluation of Discriminatory Power of the Selected Dissolution Medium
Dissolution comparison with Ezetimibe tablet (10 mg)
Dissolution condition: 0.1% SLS in 0.05M acetate buffer, pH 4.5.
USP Apparatus-II, 50 rpm, 900 mL

| | Product | | |
|---|---|---|---|
| | Zetia (10 mg) + Bempedoic | | |
| | acid (180 mg) tablet | | |
| | (Reference Product) | FDC Product | FDC Product |
| Batch/Lot No. | L015901 + N460335 | 4759-S1-096 | 4490-S1-047 |
| Time (min.) | % Drug Release (ezetimibe) | | |
| 15 | 74.7 | 67.7 | 45.5 |
| 30 | 75.6 | 70.7 | 49.2 |
| 45 | 74.6 | 71.9 | 51.5 |
| 60 | 74.3 | 73.3 | 54.0 |

The discriminatory dissolution media reflected the difference in processing for ezetimibe granulation and correlated with the expected dissolution behavior. The discriminatory power of the selected media was confirmed. The dissolution profile of Zetia (10 mg)+bempedoic acid (180 mg) (reference product) was found to be comparable with the test FDC product (batch no: 4759-S1-096).

### 4.2.3 Method Development of Discriminatory Dissolution Method for Bempedoic Acid

The QC release medium, (phosphate buffer, pH 6.8) showed dose dumping (˜90% in 15 minutes). Optimization of the surfactant concentration (0.1% to 0.45%) was performed with 1000 mL of different dissolution media using USP Apparatus II at 50 rpm.

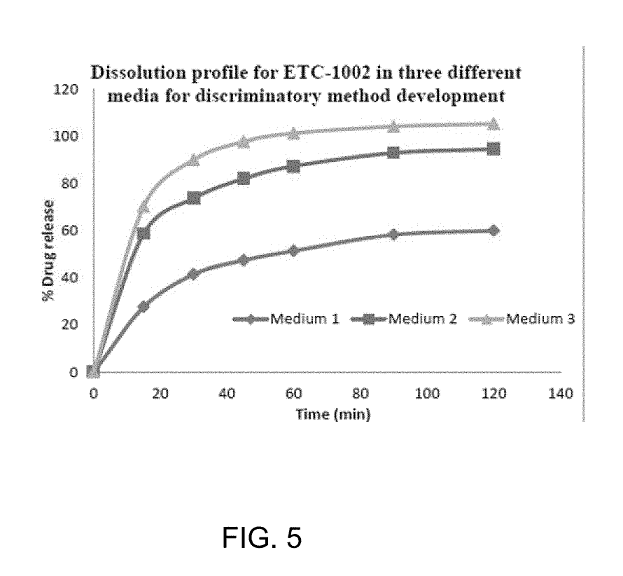

The dissolution was performed on the Zetia (10 mg)+bempedoic acid (180 mg) reference product and the dissolution data is shown in Table 11 and FIG. 5.

TABLE 11
Method Development Data for Development of
Discriminatory Dissolution Method for Bempedoic Acid

| | Medium 1: | Medium 2: | Medium 3: |
|---|---|---|---|
| | 0.1% SLS in | 0.45% SLS in | 0.2M phosphate |
| Time | acetate buffer, | acetate buffer, | buffer, |
| Points | pH 4.5, | pH 4.5 | pH 6.8 |
| (minutes) | % Drug Release (Bempedoic acid) | | |
| 15 | 27.6 | 58.8 | 70.4 |
| 30 | 41.6 | 73.9 | 90.2 |
| 45 | 47.4 | 82.2 | 97.8 |
| 60 | 51.4 | 87.5 | 101.3 |
| 90 | 58.3 | 93.1 | 104.2 |
| 120 | 60.1 | 94.7 | 105.3 |

Media Medium 1 (0.1% SLS in acetate buffer, pH 4.5) showed a slower release profile and complete recovery was not observed within 2 hrs. Medium 3 (0.2 M phosphate buffer, pH 6.8) showed dumping behavior. Medium 2 (0.45% SLS in acetate buffer, pH 4.5) showed a noticeable gradual dissolution profile for bempedoic acid. Therefore, 0.45% SLS in acetate buffer, pH 4.5, 1000 mL, 50 rpm, USP App-II was finalized as the discriminatory dissolution condition.

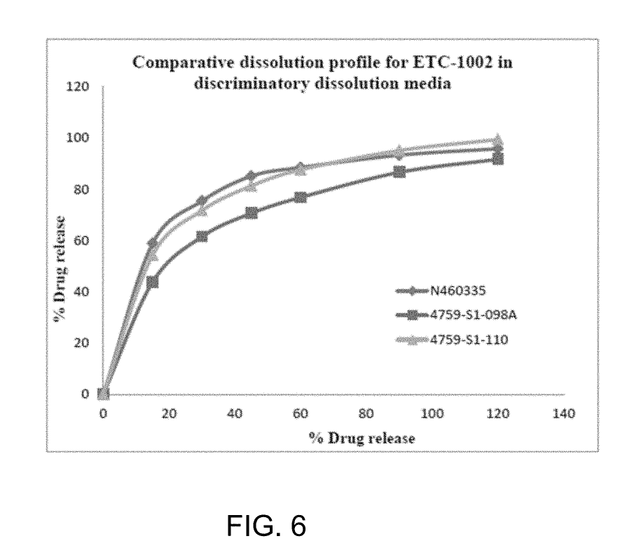

The discriminatory power of the method was demonstrated with slight change in particle size of bempedoic acid in the formulation and is shown in Table 12 and FIG. 6.

TABLE 12
Comparative Dissolution Profile of Bempedoic Acid
Tablet (Reference Product) vs. FDC-Monolayer Tablet
with Coarser and Fine Grade Bempedoic Acid
Dissolution comparison for ETC-1002
Dissolution condition: 0.45% SLS in acetate buffer, pH
4.5, 1000 mL, 50 rpm, USP App-II

| | Product | | |
|---|---|---|---|
| | | Trial with Coarser | Trial with Finer |
| | Bempedoic | Grade Bempedoic | Grade |
| | Acid Tablet | Acid | Bempedoic Acid |
| PSD of API | (Reference | % Retained/% passed | |
| #60 retained/passed | Product) | 32.0/68.0 | 11.5/88.5 |
| Batch/Lot No. | N460335 | 4759-S1-098A | 4759-S1-110 |
| Time (min.) | % Drug Release (bempedoic acid) | | |
| 15 | 58.9 | 43.8 | 54.3 |
| 30 | 75.4 | 61.5 | 71.7 |
| 45 | 85.1 | 70.6 | 81.3 |
| 60 | 88.5 | 76.8 | 87.6 |
| 90 | 93.3 | 86.6 | 95.1 |
| 120 | 95.8 | 91.7 | 99.4 |
| R | 96.5 | 94.9 | 101.8 |

| | | |
|---|---|---|
| F2 Value | 46.0 | 73.0 |

As expected the batch with coarser grade bempedoic acid (#60 retained/pass=32/68) showed lower release compared to the batch with finer grade bempedoic acid (#60 retained/pass=11.5/88.5). The difference in dissolution profile (due to change in particle size of active) reflects the discriminatory power of the selected dissolution condition.

### 5 Drug Substance

Bempedoic acid was obtained from Esperion Therapeutics, Inc. and Ezetimibe was procured from Teva API India Ltd.

5.1 Physical Properties

The compressibility indices of 32.6% and 29.60% and Hausner's ratios of 1.48 and 1.42 for bempedoic acid and ezetimibe, respectively, suggest very poor flowability of the APIs.

### 5.2 Chemical Properties:

Chemical properties are detailed in Table 14.

TABLE 14
Chemical Properties of Ezetimibe and Bempedoic Acid

| | Description | |
|---|---|---|
| Properties | Bempedoic Acid | Ezetimibe |
| Structural Formula | 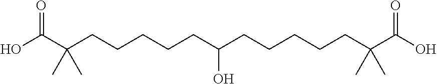 [SMILES: CC(C)(CCCCCC(O)CCCCCC(C)(C)C(=O)O)C(=O)O] | 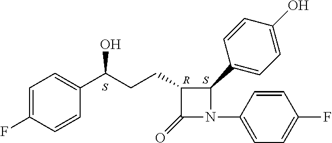 |
| Chemical Name | 8-hydroxy-2,2,14,14- | (3R,4S)-1-(4-fluorophenyl)-3-[(3S)- |
| | tetramethylpentadecanedioic acid | 3-(4-fluorophenyl)-3- |
| | | hydroxypropyl]-4- |
| | | (4-hydroxyphenyl) azetidin-2-one. |
| Molecular Formula | C19H36O5 | C24H21F2NO3 |
| Molecular Weight | 344.49 g/mol | 409.4 g/mol |
| Melting Point | 88° C.-91° C. | 164° C.-166° C. |
| pKa | 4.88, 5.60 (determined by | 9.75 (determined by |
| | potentiometric titration) | potentiometric titration) |

### 5.2.1 Polymorphism

Ezetimibe drug substance possesses different polymorphic/hydrate forms.

Anhydrous (designated as Form A)
Hydrate form (designated as Form B)

Both polymorphic forms exhibit the same physicochemical properties. For FDC pharmaceutical development, the anhydrous form of ezetimibe (Form A) was used.

Bempedoic acid is a crystalline powder with no evidence of polymorphic formation.

### 5.2.2 Chemical Stability

Forced degradation of bempedoic acid was carried out to study the impurity profile, degradation pathway, and to facilitate the development of a stability-indicating method. In addition, knowledge obtained from the forced degradation studies was used during formulation and process design and development to prevent impurities from being generated. The specified stress conditions were intended to achieve 5-20% degradation. The stressed samples were compared to the unstressed sample (control).

Forced degradation conditions and results for bempedoic acid are listed in Table 15.

TABLE 15
Summary of Forced Degradation Data for Bempedoic Acid

| | | | % Degradation | | | | | | | |
|---|---|---|---|---|---|---|---|---|---|---|
| Exposed | Assay | RRT | RRT | RRT | RRT | RRT | RRT | RRT | RRT | Total |
| to (1) | % | 0.94 | 0.97 | 1.03 | 1.04 | 1.25 | 1.28 | 1.95 | 2.29 | impurities |
| Non | 99.0 | — | — | — | — | — | — | — | — | — |
| stressed | | | | | | | | | | |
| bempedoic | | | | | | | | | | |
| acid | | | | | | | | | | |
| HCl | 98.7 | — | — | 0.06 | — | — | 1.03 | — | — | 1.09 |
| NaOH | 117.3(2) | — | — | — | 0.22 | — | — | — | 0.85 | 1.07 |
| H2O2 | 100.4 | — | — | — | — | — | — | — | | — |
| Thermal, | 97.7 | 0.07 | — | — | — | — | — | 2.09 | 0.56 | 2.72 |
| solution | | | | | | | | | | |
| 105□□C | | | | | | | | | | |
| Thermal, | 95.8 | — | 0.08 | — | 0.20 | 0.06 | — | 2.06 | — | 2.40 |
| solid state | | | | | | | | | | |
| 105° C. | | | | | | | | | | |
| Photo, solid | 99.2 | — | — | — | — | — | — | — | — | — |
| state | | | | | | | | | | |

(1) Appropriate control samples were prepared for each stressor but are not shown in above table.
(2)w/w % bempedoic acid in NaOH result differs from expected value of approximately 100% due to non-quantitative neutralization prior to injection

No significant change was observed in the purity profile of Bempedoic acid during the stress test study. This indicates that the API is stable at all studied conditions.

The summary of forced degradation data for ezetimibe are presented in Table 16.

TABLE 16
Summary of Forced Degradation Data for Ezetimibe

| | | % Degradation | | | |
|---|---|---|---|---|---|
| | | | | | Total |
| | | | EZT | | (unspecified |
| | Assay | EZT- | Cyclic | EZT- | and |
| Exposed to | (%) | Amide | ether | Keto | specified) |
| Non stressed ezetimibe | 100.0 | ND | 0.07 | ND | 0.12 |
| 5.0 mL stock solution + | 97.9 | ND | 0.09 | ND | 0.83 |
| 1 mL | | | | | |
| 1N HCl at RT for 24 hrs | | | | | |
| 5.0 mL stock solution + 0.1 | 83.1 | 1.06 | 9.23 | ND | 14.66 |
| mL 1N NaOH at RT for 30 | | | | | |
| min | | | | | |
| 5.0 mL stock solution + | 97.0 | ND | 0.75 | ND | 0.91 |
| 1 mL | | | | | |
| H2O2 30% at RT for 24 hrs | | | | | |
| 5.0 mL stock solution in Sun | 95.0 | ND | 0.40 | 0.05 | 1.57 |
| cabinet 765 W/m2 at 35° C. | | | | | |
| for 15 hrs | | | | | |
| 5.0 mL stock solution + | 93.1 | ND | 4.81 | ND | 5.67 |
| Heating at 80° C. for 30 min | | | | | |
| 21.41 mg of solid + Heating | 96.3 | ND | 0.09 | ND | 0.14 |
| at 80° C. for 24 hrs | | | | | |
| 18.48 mg of solid in Sun | 98.0 | ND | 0.06 | ND | 0.22 |
| cabinet 765 W/m2at 35° C. | | | | | |
| for 24 hrs | | | | | |

It has been observed that ezetimibe is relatively unstable particularly upon exposure to alkali (NaOH) and peroxide (H2O2). The main degradation product identified is EZT cyclic ether. In the solid form however it is stable to heat and photo irradiation. Therefore, ezetimibe is classified as sensitive to alkali and peroxide.

### 5.3 Biological Properties

Biological properties are given in Table 17.

TABLE 17
Biological Properties of Bempedoic Acid and Ezetimibe

| | Description | |
|---|---|---|
| Properties | Bempedoic Acid | Ezetimibe |
| Category | Low-density | Cholesterol |
| | lipoprotein cholesterol | absorption |
| | (LDL-C) lowering agent | inhibitors |
| Maximum | 180 mg, once a day | 10 mg, once a |
| recommended | | day with or |
| dose | | without food. |
| BCS class | BCS class II | BCS class II |
| Log partition | Octanol/water: | n-octanol: 0.1N |
| coefficient (log p) | 4.328 Octanol/pH 3.5 | HCl: 4.52 n-octanol: |
| | buffer: 4.382 | pH 7: 4.51 |

### 5.4 Risk Assessment of Drug Substance Attributes

A risk assessment of the drug substance attributes was performed to evaluate the impact of each attribute on the drug product CQAs. The relative risk ranking system used throughout the pharmaceutical development is summarized in Table 18. The outcome of the assessment and the accompanying justifications are provided in Table 19, Table 20, Table 21, and Table 22. The relative risk that each drug substance attribute was ranked as high, medium, or low. Those attributes that could have a high impact on the drug product CQAs warranted further investigation whereas those attributes that had low impact on the drug product CQAs required no further investigation.

TABLE 18
Overview of relative risk ranking system

| | |
|---|---|
| Low | Broadly acceptable risk. No further |
| | investigation is needed. |
| Medium | Risk is acceptable. Further investigation |
| | may be needed in order to reduce the risk. |
| High | Risk is unacceptable. Further |
| | investigation is needed to reduce the risk. |

TABLE 19
Risk assessment for Bempedoic Acid

| | Drug Substance Atttibutes | | | | | | | | |
|---|---|---|---|---|---|---|---|---|---|
| Drug | Solid | Particle | | | | | | | |
| product | state | size | | Moisture | Chemical | Flow | Hygro- | Total | Residual |
| CQA | form | distribution | Solubility | content | stability | properties | scopicity | impurity | solvent |
| Assay | Low | Low | Low | Low | Low | Medium | Low | Low | Low |
| Content | Low | Low | Low | Low | Low | Medium | Low | Low | Low |
| uniformity | | | | | | | | | |
| Dissolution | Low | Medium | Medium | Low | Low | Low | Low | Low | Low |
| Degradation | Low | Low | Low | Low | Low | Low | Low | Medium | Low |
| product | | | | | | | | | |

TABLE 20
Risk Assessment Justification for Impact of Drug Substance Attributes of
Bempedoic Acid on Drug Products CQA

| Drug | | |
|---|---|---|
| Substance | Drug product | |
| Attributes | CQA | Justification |
| Solid state | Assay | Drug substance solid state form does not affect tablet assay and CU. The risk is low. |
| form | Content uniformity | |
| | Dissolution | Bempedoic acid does not have any polymorphic forms. The solid state of bempedoic acid has no impact on |
| | | dissolution. The risk is low. |
| | Degradation | The solid form of BEMPEDOIC ACID used in the development is stable; it will not contribute to degradation |
| | product | product. The risk is low. |
| PSD | Assay | Bempedoic acid quantity in the formulation is more than 40%. The risk is low. |
| | Content uniformity | |
| | Dissolution | Bempedoic acid has low solubility. PSD may affect solubility and thus dissolution. The risk is medium. |
| | Degradation product | Particle size does not impact the degradation products. The risk is low. |
| Solubility | Assay | Solubility does not have an impact on assay & content uniformity. The risk is low. |
| | Content uniformity | |
| | Dissolution | Bempedoic acid has low solubility which may impact dissolution The risk is medium. |
| | Degradation product | Solubility does not affect tablet degradation products. The risk is low. |
| Moisture | Assay | Bempedoic acid is non-hygroscopic. Moisture content of the drug substances does not impact assay, CU, dissolution, |
| content | Content uniformity | and degradation products. The risk is low. |
| | Dissolution | |
| | Degradation product | |
| Chemical | Assay | Bempedoic acid is chemically stable. The impact to chemical stability of drug substance on assay, CU, dissolution |
| stability | Content uniformity | and degradation product is minimal. The risk is low. |
| | Dissolution | |
| | Degradation product | |
| Flow | Assay | Bempedoic acid has poor flow properties which can have impact on assay and CU. The risk is medium. |
| Properties | Content uniformity | |
| | Dissolution | The flowability of the drug substance is unlikely to affect its degradation pathway or solubility. The risk is low. |
| | Degradation product | |
| Hygroscopicity | Assay | Bempedoic acid is non hygroscopic hence it is unlikely to impact Assay, Content uniformity, Dissolution and |
| | Content uniformity | Degradation product of drug product. The risk is low. |
| | Dissolution | |
| | Degradation product | |
| Total | Assay | Total impurities are controlled in the drug substance specification (NMT 2.0%). Impurity limits comply with ICH |
| impurities | Content uniformity | Q3A recommendations. Within this range, process impurities are unlikely to impact assay, CU and dissolution. The |
| | Dissolution | risk is low. |
| | Degradation product | Process impurities that are potential degradants may increase overtime. The risk is medium. |
| Residual | Assay | Residual solvents are controlled in the drug substance specification and comply with USP <467>. At controlled |
| solvents | Content uniformity | levels, residual solvents are unlikely to impact assay, CU, dissolution & degradation. The risk is low. |
| | Dissolution | |
| | Degradation product | |

TABLE 21
Risk Assessment for Ezetimibe

| | Drug Substance Attributes for Ezetimibe | | | | | | | | |
|---|---|---|---|---|---|---|---|---|---|
| Drug | Solid | | | | | | | | |
| product | state | | | Moisture | Chemical | Flow | Hygro- | Process | Residual |
| CQA | form | PSD | Solubility | content | stability | properties | scopicity | impurity | solvent |
| Assay | Low | Medium | Low | Low | Low | Medium | Low | Low | Low |
| Content | Low | Medium | Low | Low | Low | Medium | Low | Low | Low |
| uniformity | | | | | | | | | |
| Dissolution | Low | High | Medium | Low | Low | Low | Low | Low | Low |
| Degradation | Low | Low | Low | Low | Medium | Low | Low | Medium | Low |
| product | | | | | | | | | |

TABLE 22
Risk Assessment Justification for Impact of Drug Substance Attributes on the
Drug Product CQAs

| Drug Substance | | |
|---|---|---|
| Attributes | Drug Product CQA | Justification |
| Solid state form | Assay | The anhydrous form of ezetimibe was used for |
| | Content uniformity | the development. Both anhydrous and hydrate |
| | Dissolution | forms show similar physical and chemical |
| | Degradation product | properties which will not affect Assay, Content |
| | | uniformity, Dissolution, Degradation product. |
| | | The risk is low. |
| PSD | Assay | A small particle size and a wide PSD may |
| | | adversely impact blend flowability. In extreme |
| | | cases, poor flowability may impact assay. The |
| | | risk is medium. |
| | Content uniformity | Particle size distribution has a direct impact on |
| | | drug substance flowability and ultimately on CU. |
| | | Improper mixing with excipients may lead to |
| | | problems in assay and content uniformity. The |
| | | risk is medium. |
| | Dissolution | Drug substance has low solubility. PSD may |
| | | affect solubility and hence the dissolution. The |
| | | risk is high. |
| | Degradation product | Particle size is unlikely to impact the degradation |
| | | products. The risk is low. |
| Solubility | Assay | Solubility is unlikely to have an impact on assay |
| | Content uniformity | & content uniformity. The risk is low. |
| | Dissolution | Ezetimibe exhibited low solubility across |
| | | physiological pH range. Drug substance |
| | | solubility does impact dissolution. The risk is |
| | | medium. |
| | Degradation product | Solubility is unlikely to affect tablet degradation |
| | | products. Thus, the risk is low. |
| Moisture content | Assay | Moisture is controlled in the drug substance |
| | Content uniformity | specification (NMT 1.5%). Thus, it is unlikely to |
| | Dissolution | impact assay, content uniformity and dissolution. |
| | Degradation product | The risk is low. |
| Chemical stability | Assay | Total impurities are controlled in the drug |
| | Content uniformity | substance specification (NMT 0.5%). Impurity |
| | Dissolution | limits comply with ICH Q3A recommendations. |
| | | Within this range, process impurities are unlikely |
| | | to impact assay, CU and dissolution. The risk is |
| | | low. |
| | Degradation product | Impurity of drug substance may impact the |
| | | formulation impurity profile. The risk is |
| | | medium. |
| Flow properties | Assay | Ezetimibe has poor flow properties which can |
| | Content uniformity | have impact on assay and CU. The risk is |
| | | medium. |
| | Dissolution | The flowability of the drug substance is unlikely |
| | Degradation product | to affect its degradation pathway or solubility. |
| | | The risk is low. |
| Hygroscopicity | Assay | Ezetimibe is hygroscopic; however, in the |
| | Content uniformity | granules LOD is controlled with a limit of NMT |
| | Dissolution | 2.0% w/w. Further, the HDPE packed with |
| | Degradation product | desiccant ensures the physical stability. The risk |
| | | is low. |
| Process impurities | Assay | Total impurities are controlled in the drug |
| | Content uniformity | substance specification (NMT 0.15%). Impurity |
| | Dissolution | limits comply with ICH Q3A recommendations. |
| | | Within this range, process impurities are unlikely |
| | | to impact assay, CU, and dissolution. The risk is |
| | | low. |
| | Degradation product | Process impurities may increase upon stability. |
| | | The risk is medium. |
| Residual solvents | Assay | Residual solvents are controlled in the drug |
| | Content uniformity | substance specification and comply with USP |
| | Dissolution | <467>. At controlled levels, residual solvents are |
| | Degradation product | unlikely to impact assay, CU, dissolution & |

### 5.5 Drug-Drug Interaction

The fixed dose combination product under development is a novel combination of two drug substances and therefore determination of compatibility of the actives with each other was considered to be critical. The compatibility of the actives was assessed through HPLC analysis of binary mixtures of the drug substances at a ratio 1:18 (ezetimibe:bempedoic acid) in the solid state. Samples were stored at 60° C. and 40° C./75% RH in both open and closed containers for 2 weeks and 4 weeks respectively. Degradation products were evaluated for open (stressed) samples of accelerated condition (40° C./75% RH) and 60° C. Assay for the samples was also carried out. Results are summarized in Table 23.

TABLE 23
Drug-Drug Compatibility Study Results

| | | EZT | | EZT 21 | | | | |
|---|---|---|---|---|---|---|---|---|
| | | cyclic | RRS | (Keto | | Total | | |
| Samples | Condition | ether | isomer | impurity) | SMUI | impurities | Assay | |
| Ezetimibe | Initial | ND | ND | 0.03 | 0.03 | 0.06 | 99.3 | |
| | 2W 60° | 0.12 | ND | 0.04 | 0.17 | 0.33 | 97.6 | |
| | 4W 40° C./ | 0.90 | ND | 0.38 | 0.19 | 2.34 | 99.5 | |
| | 75% | | | | | | | |
| | RH Open | | | | | | | |
| Bempedoic | Initial | No unspecified impurity generated | | | | | 101.7 | |
| acid | 2W 60° | | | | | | 100.2 | |
| | 4W 40° C./ | | | | | | 103.7 | |
| | 75% | | | | | | | |
| | | | | | | | | Bempedoic |
| | | | | | | | EZT | Acid |
| Ezetimibe | Initial | ND | ND | ND | ND | ND | 100.0 | 101.3 |
| + | 2W 60° | ND | ND | 0.05 | ND | 0.05 | 103.8 | 97.6 |
| Bempedoic | 4W 40° C./ | ND | ND | 0.04 | 0.13 | 0.17 | 99.0 | 98.2 |
| acid | 75% | | | | | | | |

There was no significant increase in any of the impurities for each of the actives in presence of each other under accelerated conditions and the assay was found to be within the acceptable limits. Therefore, bempedoic acid and ezetimibe can be considered to be chemically compatible.

### 5.6 Excipients

The excipients used in the combination drug product were selected based on the excipients used in the individual reference products, excipient compatibility studies, and prior use in approved drug products. The drug-excipient compatibility studied combined both APIs with selected excipients.

### 5.6.1 Drug-Excipient Compatibility Study

Excipients-drug substance compatibility was assessed using HPLC analysis of ternary mixtures of excipients and both APIs together at a required ratio in the solid state. Samples were stored at 60° C. and 40° C./75% RH in both open and closed containers for 2 weeks and 4 weeks respectively. Common excipients functioning as filler, disintegrant, binder, and lubricant were evaluated. Degradant level (related substance) for each API was assessed using HPLC analysis to quantify the degradations in case of any incompatibility. Degradation products were evaluated for open (stress) samples at accelerated conditions (40° C./75% RH) and 60° C. Samples held closed at accelerated condition (40° C./75% RH) were evaluated when there was a significant increase in degradation observed in the open (stress) conditions. Assay for the samples was also carried out. Results are summarized in Table 24.

Table 25 describes the compatibility study of both actives together with the excipients used in the formulation.

TABLE 24
Batches for drug-excipient compatibility study

| Sr. | | | |
|---|---|---|---|
| No. | Ingredients | Batch No. | Ratio |
| 1 | (Ezetimibe + Bempedoic acid) + | 4490-CO-014-O | 1:1 |
| | Pharmatose 200M | | |
| 2 | (Ezetimibe + Bempedoic acid) + Avicel | 4490-CO-014-P | 1:1 |
| | PH-102 | | |
| 3 | (Ezetimibe + Bempedoic acid) + Kollidon | 4490-CO-014-Q | 1:0.5 |
| | 30 | | |
| 4 | (Ezetimibe + Bempedoic acid) + HPC-L | 4490-CO-014-R | 1:0.5 |
| 5 | (Ezetimibe + Bempedoic acid) + | 4490-CO-014-T | 1:0.5 |
| | Kolliphor SLS Fine | | |
| 6 | (Ezetimibe + Bempedoic acid ) + Primojel | 4490-CO-014-V | 1:0.5 |
| 7 | (Ezetimibe + Bempedoic acid) + Aerosil | 4490-CO-014-W | 1:0.1 |
| | 200P | | |
| 8 | (Ezetimibe + Bempedoic acid) + | 4490-CO-014-X | 1:0.1 |
| | magnesium stearate | | |
| 9 | (Ezetimibe + Bempedoic acid) + | 4490-CO-014-Z | 1:0.25 |
| | Opadry white (85F18422) | | |

TABLE 25
Drug-Excipient Compatibility Study Results

| | | EZT | | EZT 21 | | | Assay | |
|---|---|---|---|---|---|---|---|---|
| | | cyclic | RRS | (Keto | | Total | | Bempedoic |
| Samples | Condition | ether | isomer | impurity) | SMUI | impurities | EZT | Acid |
| Ezetimibe + | Initial | ND | ND | ND | ND | ND | 103.1 | 98.2 |
| Bempedoic | 2W 60° Open | ND | ND | 0.04 | ND | 0.04 | 97.0 | 96.8 |
| acid + | 4W 40° C./ | ND | ND | 0.05 | 0.04 | 0.09 | 97.8 | 99.9 |
| Pharmatose | 75% RH Open | | | | | | | |
| 200M) | | | | | | | | |
| Ezetimibe + | Initial | ND | ND | ND | ND | ND | 95.2 | 101.6 |
| Bempedoic | 2W 60° Open | ND | ND | 0.04 | 0.03 | 0.07 | 97.4 | 96.3 |
| acid + Avicel | 4W 40° C./ | ND | ND | 0.04 | 0.02 | 0.06 | 99.0 | 99.4 |
| PH-102 | 75% RH Open | | | | | | | |
| Ezetimibe + | Initial | ND | ND | 0.03 | ND | 0.03 | 98.0 | 98.6 |
| Bempedoic | 2W 60° Open | ND | ND | 0.08 | ND | 0.08 | 99.9 | 95.0 |
| acid + | 4W 40° C./ | 0.13 | ND | 0.16 | 0.02 | 0.35 | 99.3 | 95.7 |
| Kollidon 30 | 75% RH Open | | | | | | | |
| Ezetimibe + | Initial | ND | ND | 0.03 | ND | 0.03 | 101.1 | 95.9 |
| Bempedoic | 2W 60° Open | 0.11 | ND | 0.15 | 0.10 | 0.039 | 99.4 | 98.2 |
| acid + HPC- | 4W 40° C./ | 0.34 | ND | 0.06 | 0.2 | 0.46 | 101.1 | 96.4 |
| L | 75% RH Open | | | | | | | |
| | 4W 40° C./ | ND | ND | ND | ND | 0.02 | Not done | |
| | 75% RH closed | | | | | | | |
| Ezetimibe + | Initial | ND | ND | 0.03 | ND | 0.03 | 99.1 | 101.6 |
| Bempedoic | 2W 60° Open | ND | ND | 0.04 | ND | 0.04 | 96.5 | 100.7 |
| acid + | 4W 40° C./ | ND | ND | 0.04 | ND | 0.04 | 97.4 | 99.5 |
| Kolliphor | 75% RH Open | | | | | | | |
| SLS Fine | | | | | | | | |
| Ezetimibe + | Initial | ND | ND | 0.03 | ND | 0.03 | 97.3 | 102.3 |
| Bempedoic | 2W 60° Open | ND | ND | 0.05 | ND | 0.05 | 98.1 | 101.9 |
| acid + | 4W 40° C./ | ND | ND | 0.04 | ND | 0.04 | 98.3 | 99.8 |
| Primojel | 75% RH Open | | | | | | | |
| (Batch No | | | | | | | | |
| 4490-CO- | | | | | | | | |
| 014-V) | | | | | | | | |
| Ezetimibe + | Initial | ND | ND | ND | ND | ND | 95.9 | 101.1 |
| Bempedoic | 2W 60° Open | ND | ND | 0.07 | 0.04 | 0.11 | 104.4 | 98.5 |
| acid + | 4W 40° C./ | ND | ND | 0.04 | 0.04 | 0.08 | 97.7 | 103.6 |
| Aerosil 200P | 75% RH Open | | | | | | | |
| Ezetimibe + | Initial | ND | ND | ND | ND | ND | 99.8 | 101.1 |
| Bempedoic | 2W 60° Open | ND | ND | 0.06 | ND | 0.06 | 98.7 | 99.5 |
| acid + | 4W 40° C./ | ND | ND | 0.05 | 0.02 | 0.07 | 99.1 | 100.9 |
| Magnesium | 75% RH Open | | | | | | | |
| stearate | | | | | | | | |
| Ezetimibe + | Initial | ND | ND | 0.03 | ND | 0.03 | 102.9 | 104.8 |
| Bempedoic | 2 W 60° C. | 9.05 | ND | 0.13 | 1.38 | 11.06 | 91.7 | 100.7 |
| acid + Open | | | | | | | | |
| Opadry white | 4W 40° C./ | 6.26 | ND | 6.32 | 0.95 | 7.66 | 91.8 | 101.2 |
| (85F18422) | 75% RH Open | | | | | | | |
| | 4W 40° C./ | 6.27 | ND | 0.08 | 0.11 | 0.29 | — | |
| | 75% RH Closed | | | | | | | |

No significant change in appearance was observed for Bempedoic acid; however an increase in cyclic ether impurity was observed for open Ezetimibe at 40° C./75% RH. The combination of high temperature and humidity could have triggered this degradation. However, in the presence of Bempedoic acid and excipient, Ezetimibe is diluted and the impact of heat and humidity could be reduced.

An increase in the Ezetimibe cyclic ether was observed when combined with HPC-L in an open container maintained for 4 W at 40° C./75% RH. However, this drug-excipient combination was found to be compatible at 4 W 40° C./75% RH when in a closed container. HPC-L is used in the granulation of Bempedoic acid and is not in direct contact with Ezetimibe; therefore, the excipient was selected in the final formulation.

Assay values of both Ezetimibe and Bempedoic acid were comparable to the initial samples, except for Opadry white where a drop in the assay value of Ezetimibe was observed along with an increase in the impurity levels. With increase in impurities at all conditions, the excipient, Opadry white (85F18422), was found to be incompatible. It is important to note that the reference product for Bempedoic acid mono product is a coated tablet whereas the reference product, Zetia, is uncoated tablet. The target for the fixed dose combination is a coated tablet. Therefore, an additional compatibility study with another Opadry system, without polyethylene glycol and polyvinyl alcohol (main component of studied Opadry) was proposed.

### 5.6.2 Selection of Excipient Grade

Based on the results of the drug-excipient compatibility studies, excipients identical to the reference product formulations were selected for the development of the fixed dose combination product. The levels of excipients to be used in the formulation were studied in subsequent formulation development studies.

### Lactose Monohydrate (Pharmatose® 200M):

Lactose monohydrate is commonly used as filler. Usually, fine grades of lactose are used in the preparation of tablets using a wet-granulation method. Pharmatose® 200M, a lactose monohydrate, from DFE Pharma was selected. Particle size distribution data of Pharmatose 200M shows more than 90% particles are less than 100 μm size and a bulk density of 0.56 g/cm3.

### Microcrystalline Cellulose (Avicel® PH-102):

Microcrystalline cellulose is widely used as filler in direct compression and roller compaction. It undergoes plastic deformation during compaction since it is fibrous and ductile. Microcrystalline cellulose (Avicel® PH-102) is used in the current bempedoic acid tablet formulation as a diluent with larger particle size (100 μm) which helps to provide better blend flow properties. The moisture content is 3.0-5.0% and the bulk density is 0.28-0.33 g/cc.

### Hydroxypropyl Cellulose (HPC-L):

Hydroxypropyl cellulose is a partially-substituted poly (hydroxypropyl) ether of cellulose. Hydroxypropyl cellulose is commercially available in a number of different grades that have various solution viscosities. The grade to be used in the formulation is the regular grade fine powder with a range in viscosity range from 6.0 to 10.0 mPa·s, which is mostly used as a binder in tablet dosage form.

### Povidone K30 (Kollidon® 30):

Povidone (Kollidon® 30) is a medium molecular weight grade of povidone with a K value of 27.0-32.1. It is versatile and widely used as a binder in tablets and granules. In the current formulation, an aqueous solution of povidone is used as a binder in the top spray granulation process for ezetimibe.

### Sodium Lauryl Sulfate (Kolliphor® SLS Fine):

Sodium lauryl sulfate is an anionic surfactant employed in a wide range of oral pharmaceutical formulations for the dissolution improvement of insoluble drug molecules.

Sodium lauryl sulfate is used as a solubilizer in concentrations greater than critical micelle concentration i.e. >0.0025%. It is used as wetting agent, effective in both alkaline and acidic conditions. In the current formulation it is used as a dissolution enhancer for ezetimibe.

### Sodium Starch Glycolate (Primojel®):

Sodium starch glycolate is a white or almost white free-flowing very hygroscopic powder. It is widely used in oral pharmaceuticals as a disintegrant in tablet manufacturing. Disintegration occurs by rapid uptake of water followed by rapid and enormous swelling of tablets containing sodium starch glycolate.

The effectiveness of many disintegrants is affected by the presence of hydrophobic excipients, such as lubricants. Increasing the tablet compression pressure appears to have no effect on disintegration time.

### Colloidal Silicon Dioxide (Aerosil® 200P):

Colloidal silicon dioxide (Aerosil® 200 Pharma), a commercial grade of colloidal silicon dioxide manufactured by Evonik, was used as a glidant in the current bempedoic acid tablet formulation.

### Magnesium Stearate:

Magnesium stearate, manufactured by Avantor using a vegetable origin, was used as lubricant in the current formulation. It has particle size specification of 99 to 100% w/w passing through #325 sieve (ASTM, 45 μm) and LOD<5.0% w/w.

### Opadry AMB II 88A180040 White:

Opadry AMB II 88A180040 is a polyvinyl acetate (PVA) based non-functional film coating system with glycerol monocaprylocaprate type 1, sodium lauryl sulfate, titanium dioxide and talc used for the aesthetic appearance of the tablets.

Table 26 and Table 27 summarize the grade excipients selected for the proposed formulation and their IID limits.

TABLE 26
IID Limits of Excipients in Proposed FDC Formulations

| | | Current | | Route |
|---|---|---|---|---|
| | | formula | IID | specified in |
| Ingredients | Specification | (mg/unit) | Limit | IID listing |
| Ezetimibe | IH | 10.00 | — | — |
| Bempedoic acid | IR | 180.00 | — | — |
| Lactose monohydrate | USP/NF | 75.00 | 587.44 | Tablet, film |
| (Pharmatose ® 200M) | | | | coated |
| Microcrystalline cellulose | NF/Ph. Eur | 60.60 | 563.50 | Tablet, film |
| (Avicel ® PH-102) | | | | coated |
| Sodium starch glycolate | USP/NF/Ph. | 27.0 | 90.00 | Tablet, film |
| (Primojel ®) | Eur | | | coated |
| Povidone K30 (Kollidon ® 30) | USP/NF/Ph. | 1.00 | 80.00 | Tablet |
| | Eur | | | |
| Sodium lauryl sulfate (Kolliphor ® | USP/NF/Ph. | 2.00 | 12.00 | Tablet, film |
| SLS Fine) | Eur | | | coated |
| Colloidal silicon dioxide (Aerosil ® | USP/NF/Ph. | 4.00 | 33.00 | Tablet, film |
| 200P) | Eur | | | coated |
| Hydroxypropyl cellulose (HPC-L) | EP | 12.00 | 52.50 | Tablet, film |
| | | | | coated |
| Magnesium stearate | NF | 4.00 | 40.00 | Tablet, coated |

| | | | |
|---|---|---|---|
| Total weight of core FDC tablet | 375.6 | NA | NA |

| | | | |
|---|---|---|---|
| Opadry AMB II 88A180040 white | IH | 9.4 | Refer to Table 25 |

| | | | |
|---|---|---|---|
| Total weight of coated FDC tablet | 385.0 | NA | NA |

TABLE 27
Composition of Opadry AMB II 88A180040 White

| | | | | | Route |
|---|---|---|---|---|---|
| | | Composition | mg/ | IID Limit | specified in |
| Ingredients | Quality standard | (% w/w) | tablet | (mg) | IID listing |
| Polyvinyl alcohol | USP and Ph.Eur. | 37.0 | 3.48 | 20.0 | Tablet, film |
| part. hydrolyzed | | | | | coated |
| Talc | USP and Ph.Eur. | 31.0 | 2.91 | 320.75 | Tablet, coated |
| Titanium dioxide | USP and Ph.Eur. | 25.0 | 2.35 | 10.57 | Tablet, coated |
| Glycerol | Ph.Eur. | 4.0 | 0.38 | 62.2 | Capsule, |
| monocaprylocaprate | | | | | gelatin coated |
| Type 1 | | | | | |
| Sodium lauryl | NF and Ph.Eur. | 3.0 | 0.28 | 12.0 | Tablet, film |
| sulfate | | | | | coated |

### 6 Drug Product

### 6.1 General Information

The general information regarding drug product is given in Table 28.

TABLE 28
Drug product general information

| | |
|---|---|
| Non-Proprietary Name | Bempedoic acid and ezetimibe |
| of Drug substance | |
| Dosage Form | Immediate release tablets |
| Strength (s) | Bempedoic acid-180 mg and ezetimibe-10 |
| | mg |
| Route of Administration | Oral |
| Proposed Indication (s) | Treatment of hypercholesterolemia |

### 6.2 Formulation Development

Compatibility studies revealed that both bempedoic acid and ezetimibe were chemically compatible with each other and therefore it was possible to develop a monolayer tablet. However, the individual APIs will be granulated separately for the following reasons.

Ezetimibe exhibits poor aqueous solubility across physiological pH and therefore a surfactant, sodium lauryl sulfate (SLS), was employed in Zetia formulation. The same is preferred in the combination product to achieve bioequivalence. Bempedoic acid, however, despite having pH dependent solubility, does not require a surfactant as established in the mono product already developed by Esperion. Its increased solubility at high pH ensures dissolution and absorption in-vivo.

Bempedoic acid is sticky and has poor flowability. This aspect requires certain process steps and/or excipients that may not be suitable for ezetimibe.

### Formulation Options:

Compatibility studies ruled out any potential chemical interaction between the two APIs; however, it may be still possible that some physical interaction may lead to compromised dissolution. Additionally, the presence of SLS in a monolayer formulation may alter the dissolution of bempedoic acid and its absorption. Therefore, it was decided to develop following two formulations:

FDC-Monolayer, with both actives (in the form of separate granules) compressed into a single layer tablet, and
FDC-Bilayer, with both granules compressed into a two layer tablet with actives present in separate layers.

### 6.2.1 Initial Risk Assessment of the Formulation Variables

An overall risk assessment was performed on the impact of the drug product formulation to the drug product CQAs. Each formulation component that had a high risk of impacting a drug product CQA was further evaluated to reduce the risk. The initial risk assessment of formulation variables is presented in Table 29 and justifications for the risk assignment are presented in Table 30.

TABLE 29
Initial Formulation Risk Assessment of Different Variables

| | Formulation variables of FDC product | | | | | | |
|---|---|---|---|---|---|---|---|
| | | | | | | Ezetimibe | |
| | | | | Ezetimibe | Ezetimibe | granules | |
| | Bempedoic | | | granules | granules | Super- | |
| Drug | Acid | | Bempedoic | Surfactant | Binder | disintegrant | |
| product | Flow | Ezetimibe | Acid | concen- | concen- | concen- | Coating |
| CQA | property | PSD | Granules | tration | tration | tration | system |
| Assay | Medium | Medium | Medium | Low | Low | Low | Low |
| Content | Medium | Medium | Medium | Low | Low | Low | Low |
| uniformity | | | | | | | |
| Dissolution | Low | High | Low | High | Medium | Medium | Low |
| Degradation | Low | Low | Low | Low | Low | Low | Medium |
| product | | | | | | | |

TABLE 30
Justification of Initial Risk Assessment of Formulation Variables

| Drug Substance | Drug product | |
|---|---|---|
| Attributes | CQA | Justification |
| Bempedoic Acid | Assay | Bempedoic acid has poor flow properties which can have |
| Flow Properties | Content | impact on assay and CU. The risk is medium. |
| | uniformity | |
| | Dissolution | The flowability of the drug substance is unlikely to affect |
| | Degradation | its degradation pathway or solubility. The risk is low. |
| | product | |
| Ezetimibe PSD | Assay | A small particle size and a wide PSD may adversely |
| | | impact blend flowability. In extreme cases, poor |
| | | flowability may impact assay. The risk is medium. |
| | Content | Particle size distribution has a direct impact on drug |
| | uniformity | substance flowability and ultimately on CU. Improper |
| | | mixing with excipients may lead to problems in assay and |
| | | content uniformity. The risk is medium |
| | Dissolution | Drug is BCS class II compound having low solubility. PSD |
| | | may affect solubility and hence the dissolution. The risk is |
| | | high. |
| | Degradation | Particle size is unlikely to impact the degradation products. |
| | product | The risk is low. |
| Bempedoic acid | Assay | The poor flow properties of bempedoic acid may result |
| granules | Content | into granules that that can have impact on overall assay |
| | uniformity | and CU. The risk is medium. |
| | Dissolution | The flowability of the drug substance is unlikely to affect |
| | Degradation | its degradation pathway or solubility. The risk is low. |
| | product | |
| Ezetimibe | Assay | Surfactant concentration does not affect tablet assay and |
| granules | | CU. |
| | Content | |
| | uniformity | |
| | Dissolution | Ezetimibe is BCS class II compounds having low |
| | | solubility. For achieving bioequivalence, dissolution of |
| | | drug substances has to be improved. The risk is high for |
| | | the ezetimibe fraction of the FDC product. |
| | Degradation | Proposed surfactant is compatible with active. The risk is |
| | product | low. |
| Ezetimibe | Assay | Binder concentration does not affect tablet assay and CU. |
| granules Binder | Content | The risk is low. |
| concentration | uniformity | |
| | Dissolution | Binder concentration may affect the release profile. The |
| | | risk is medium. |
| | Degradation | Proposed binder is compatible with both the APIs hence it |
| | product | does not affect the degradation of drug product. The risk is |
| | | low. |
| Ezetimibe | Assay | Superdisintegrant concentration does not affect tablet assay |
| granules | Content | and CU. The risk is low. |
| Superdisintegrant | uniformity | |
| concentration | Dissolution | Superdisintegrant concentration determines the |
| | | disintegration time and pattern of tablet. High |
| | | disintegration time may affect dissolution profile of FDC |
| | | product. The risk is medium. |
| | Degradation | Proposed superdisintegrant is compatible hence it does not |
| | product | affect the degradation of drug product. The risk is low. |
| Coating System | Assay | Coating formulation system will not impact assay or |
| | Content | content uniformity. The risk low. |
| | uniformity | |
| | Dissolution | The selected coating system is removed immediately |
| | | during dissolution. The risk is low. |
| | Degradation | The selected coating system contains PEG and PVA which |
| | product | may interact with the API impact stability and increase the |
| | | impurity. The risk is medium. |

### 6.2.2 Formulation Development Studies

Formulation development focused on evaluation of the high and medium risk formulation variables as identified in the initial risk assessment shown in Table 28. The development was conducted in four stages. The first study optimized the bempedoic acid granulation process. The second study evaluated the impact of the level of povidone in ezetimibe granules on the drug product CQAs by OFAT (one factor at a time). The third study finalized the process for incorporation of sodium lauryl sulfate in the ezetimibe component of the drug product. The fourth study selected an appropriate coating system for the compressed tablet comprising bempedoic acid and ezetimibe.

TABLE 31
Ezetimibe Granules Formulation Development Equipment

| | | |
|---|---|---|
| Sr. No. | Process Step | Equipment |

Ezetimibe Granulation (Top spray granulation)

| | | |
|---|---|---|
| 1. | Co-sifting of | #40 mesh screen |
| | intragranular | |
| | materials | |
| 2. | Dry mixing | Fluid bed processor (Miniglatt) |
| 3. | Binder preparation | Homogenizer |
| | | Magnetic stirrer |
| 4. | Wet granulation | Fluid bed processor (Miniglatt) |
| 5. | Drying | Fluid bed processor (Miniglatt) |
| | | at 40-55° C. |
| 6. | Milling and sizing | Sifting the granules through |
| | | #30 mesh screen |
| 7. | Co-sifting of | #30 mesh screen manually |
| | extragranular | |
| | material | |

Bempedoic Acid Granulation (Wet granulation)

| | | |
|---|---|---|
| 1. | Co-sifting of | #40 mesh screen |
| | intragranular | 5 L Double cone blender |
| | materials | |
| 2. | Dry mixing | Rapid mixer granulator 2 L |
| 3. | Binder preparation | Magnetic stirrer |
| 4. | Wet granulation | Rapid mixer granulator 2 L |
| 5. | Drying | Retsch dryer |
| 6. | Milling and sizing | Sifting the dried granules through |
| | | #30 mesh screen |
| 7. | Co-sifting of | #30 mesh screen manually |
| | extragranular | |
| | material | |

Compression and Coating

| | | |
|---|---|---|
| 1. | Blending and | Double cone blender 5 L |
| | lubrication | |
| 2. | Compression | Rimek compression machine 12 station |
| | | Double rotary compression machine, |
| | | 28 station (Cadmach) |
| 3. | Coating | Coating machine ideal cure R & D |
| | | Coater (Gans Coater) |

### Process Selection

The development of the fixed dose combination of bempedoic acid and ezetimibe was attempted by fabrication of immediate release tablets in line with reference products. Both APIs were granulated separately using wet granulation. This approach ensured that ezetimibe was treated with SLS to improve solubility and bempedoic acid was treated with colloidal silicon dioxide to avoid sticky behavior. The initial development work was carried out using a monolayer approach.

### Example 2

### Formulation Development Study #1: Bempedoic Acid Granules Composition and Process Selection

The goal of Formulation Development Study #1 was to select the bempedoic acid granule composition and process of manufacturing based on a study to reduce the stickiness of the API.

Bempedoic acid exhibited poor flow and sticking during granulation and compression. The sticking of the API was prevented by creating a physical barrier between the API and the contact surface. This was achieved by coating the API with material having high surface area. Colloidal silicon dioxide was selected; it has small particle size and large specific surface area. In the preliminary trials this approach was found to be promising and therefore additional trials were carried out to optimize the concentration of colloidal silicon dioxide and process for this surface treatment.

Optimization trials were initiated using the formulation and process received from Esperion. Table 32 depicts the formula used for bempedoic acid granulation and Table 33 describes the trial batches to eliminate sticking behavior.

TABLE 32
Formula of Bempedoic Acid Granules

| Sr. | | mg/unit Batch |
|---|---|---|
| No. | Ingredients | No: 4490-S1-024 |
| | Intra-granular fraction | |
| 01 | Bempedoic acid (Non-GMP) | 180.00 |
| 02 | Microcrystalline cellulose | 51.00 |
| | (Avicel ® PH-102) | |
| 03 | Lactose monohydrate | 30.00 |
| | (Pharmatose ® 200M) | |
| 04 | Sodium starch glycolate (Primojel ®) | 14.00 |
| 05 | Hydroxypropyl cellulose (HPC-L) | 12.00 |
| 06 | Purified water | q.s. |
| Total weight of granules | | 287.00 |
| Extra-granular fraction | | |
| 07 | Magnesium stearate | 4.5 |
| 08 | Sodium starch glycolate (Primojel ®) | 7.0 |
| 09 | Colloidal silicon dioxide (Aerosil ® 200P) | 1.5 |

| | |
|---|---|
| Weight of tablet (bempedoic acid fraction) | 300.00 |

TABLE 33
Trial Batches to Eliminate Sticking of Bempedoic
Acid Tablets During Compression

| | 4490-S1-024 | | | |
|---|---|---|---|---|
| | (Esperion | 4490- | 4490- | 4759- |
| Batch No./Parameter | process) | S1-030 | S1-042 | S1-096 |
| Concentration | 1.5 | 6.5 | 4.0 | 4.0 |
| of Aerosil ® | | | | |
| 200P (mg/tablet) | | | | |
| Duration of | 10 | 30 | 30 | 45 |
| treatment (mins) | | | | |
| Manufacturing | Wet | Wet | Wet | Wet |
| process used | granulation | granulation | granulation | granulation |

Sticking behavior could be significantly reduced by treating with colloidal silicon dioxide at increasing concentrations prior to granulation. Furthermore, maintaining the level of colloidal silicon dioxide and increasing the duration of the surface treatment from 30 min to 45 mins, resulted in satisfactory processing parameters without any sticking during granulation or compression processes. The final composition of bempedoic acid granules is listed in Table 34.

TABLE 34
Final Composition of Bempedoic Acid
Granules (Batch No: 4759-S1-093)

| Sr. No. | Ingredients | mg/unit |
|---|---|---|
| 1 | Bempedoic acid | 180.00 |
| 2 | Colloidal silicon dioxide (Aerosil ® 200P) | 3.50 |
| 3 | Microcrystalline cellulose (Avicel ® PH-102) | 09.60 |
| 4 | Hydroxypropyl cellulose (HPC-L) | 12.00 |
| | Total weight of bempedoic acid granules | 205.10 |

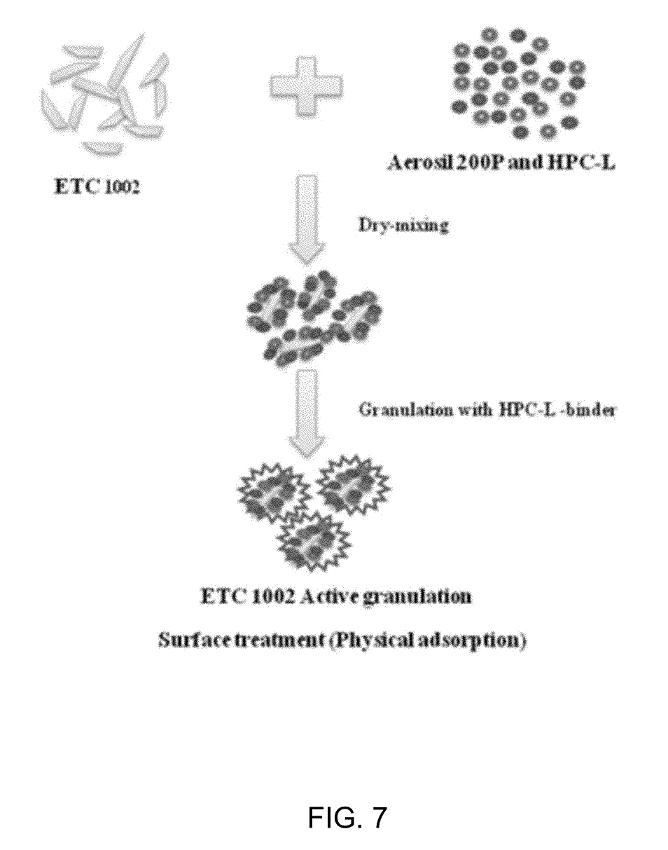

Table 35 provides the process parameters selected for further development work. A depiction of the treatment process is shown in FIG. 7.

TABLE 35
Process Parameters for Bempedoic Acid Granulation

| Batch No: 4759-S1-093 | | Batch No: 2000 tablets |
|---|---|---|
| Sr. No. | Process Step | Parameter |
| 1. | Dry mixing time | 45 minutes |
| 2. | Binder addition time | 1.5 minutes |
| 3. | Kneading time | 2.5 minutes |
| 4. | Wet sizing | Through #18 mesh |
| 5. | Drying temperature | 45° C. |
| 6. | LOD of dried granules | NMT 1% at 50° C. |
| 7. | Sifting of dried granules | #30 mesh sieve |

### Example 3

### Formulation Development Study #2: Optimization of Ezetimibe Granules:

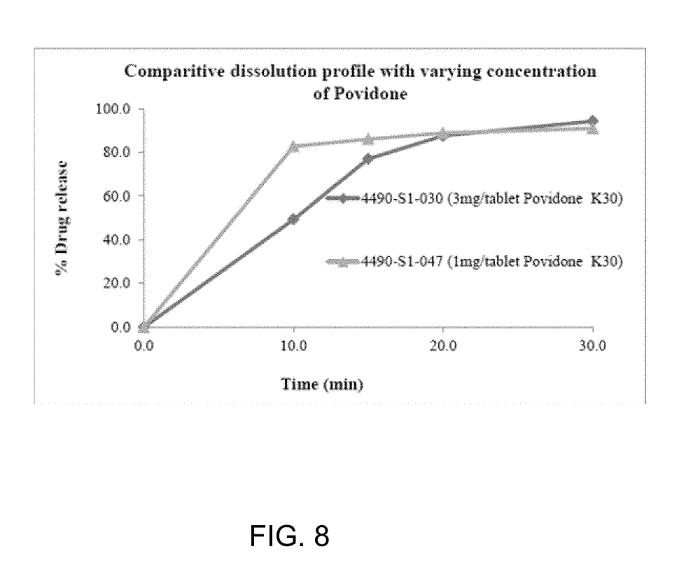

The goal of Formulation Development Study #2 was to optimize the concentration of binder used for the granulation of ezetimibe. Wet granulation was used for the preparation of ezetimibe granules. Binder solution, povidone with SLS, and ezetimibe were used for the granulation of dry-mix powder blend. One trial with a higher concentration of povidone (3 mg/tablet) and another with a lower concentration (1 mg/tablet) were performed to observe the impact of binder concentration on dissolution. Ezetimibe granules were mixed with Bempedoic acid granules separately and compressed into a monolayer tablet for both trials. A summary of the formulations is provided in Table 36. The dissolution profiles were studied in QC media (with 0.45% SLS) and are shown in Table 37 and FIG. 8.

TABLE 36
Formula of Ezetimibe Granules with
Varying Concentration of Binder

| Ingredients | mg/unit | |
|---|---|---|
| Ezetimibe granulation | 4490-S1-030 | 4490-S1-047 |
| Ezetimibe | 10.00 | 10.00 |
| Lactose monohydrate (Pharmatose ® 200M) | 50.00 | 50.00 |
| Microcrystalline cellulose (Avicel ® PH-102) | 19.00 | 21.00 |
| Sodium starch glycolate (Primojel ®) | 6.00 | 6.00 |
| Binder solution | | |
| Povidone (Kollidon ® 30) | 3.00 | 1.00 |
| Sodium lauryl sulfate (Kolliphor ® SLS Fine) | 2.00 | 2.00 |
| Purified water | q.s. | q.s. |
| Total weight of granules | 90.00 | 90.00 |

TABLE 37
Dissolution of Ezetimibe with Varying Concentration of Binder

| | 4490-S1-030 (3 mg/tablet | 4490-S1-047 (1 mg/tablet |
|---|---|---|
| Time | povidone K30) | povidone K30) |
| (mins) | % Drug Release | % Drug Release |
| 0.0 | 0.0 | 0.0 |
| 10.0 | 49.4 | 82.7 |
| 15.0 | 77.0 | 86.0 |
| 20.0 | 87.5 | 88.8 |
| 30.0 | 94.2 | 90.9 |

### Example 4

Formulation Development Study #3: Ezetimibe Granules—Optimization of SLS Incorporation Process:

The sodium lauryl sulfate concentration was estimated from the reference product, Zetia, using titration. Zetia was measured to contain 1.8 mg (˜2.0 mg) SLS per tablet. The same concentration of surfactant was considered for the ezetimibe granulation process to achieve a matching dissolution profile.

Initially, ezetimibe granules were prepared by wet granulation. The granulation process included dry-mixing ezetimibe with diluents, MCC and lactose, and superdisintegrant, SSG, followed by granulation with a binder solution containing povidone K30 and sodium lauryl sulfate as surfactant in purified water. However, the dissolution profile obtained was slower than the Zetia dissolution profile. Therefore, a series of process modification trials were carried out.

Co-Sifting of Ezetimibe with Hydrophilic Excipients (Batch No: 4759-S1-064)

Ezetimibe was co-sifted with hydrophilic excipients, lactose monohydrate (Pharmatose® 200M), Polyvinyl pyrrolidone (Kollidon® 30), through #50 mesh to reduce hydrophobicity. The blend was then granulated with a binder solution containing SLS. Ezetimibe granules were then blended with extra granular excipients and compressed into tablets. This trial did not yield the desired improvement in dissolution.

Homogenization of Binder Solution with Ezetimibe Followed by RMG Granulation (4759-S1-065)
When granulated with SLS, the surfactant was distributed over the entire mass, including the excipient. As a result, the desired improvement in dissolution was not achieved. To provide adequate contact between ezetimibe and SLS, ezetimibe was added to the binder solution and then homogenized for 30 minutes to achieve uniform dispersion. This dispersion was then granulated with the excipient blend. The granules were then blended with extra granular excipients and compressed into a tablet. Though the dissolution profile improved, it was not equivalent to the dissolution profile of Zetia.
Homogenization of Binder Solution with Ezetimibe Followed by Top Spray Granulation (Batch No: 4759-S1-094)

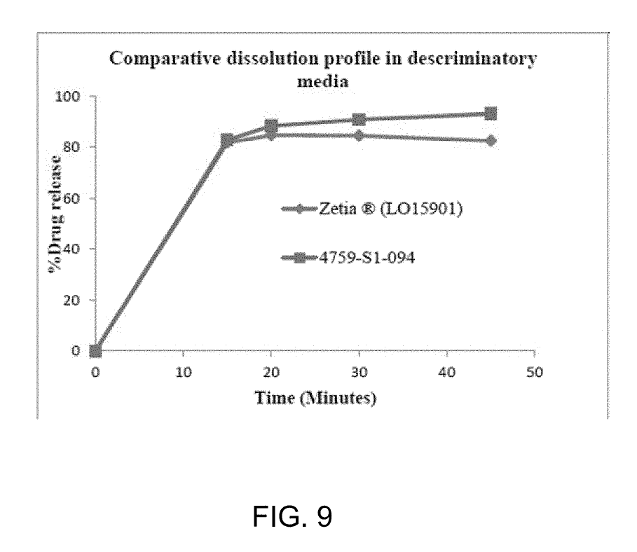

To further improvement the dissolution profile, the homogenized binder solution was incorporated with top spray granulation using a fluid bed processer (FBP) instead of rapid mixer granulator. The granules were subsequently blended with extra granular excipients and compressed into tablets. The dissolution in discriminatory dissolution media obtained is shown in Table 38 and FIG. 9.

TABLE 38
Dissolution Profile in Discriminatory Dissolution Medium

| | % Drug release | |
|---|---|---|
| Time(min) | Zetia (10 mg) (LO15901) | 4759-S1-094 |
| 0 | 0 | 0 |
| 15 | 81.8 | 82.9 |
| 20 | 84.8 | 88.4 |
| 30 | 84.6 | 90.9 |
| 45 | 82.5 | 93.1 |

### Process Parameters for Ezetimibe Granulation (Top Spray Method):

The process parameters listed in Table 39 were employed for ezetimibe granulation:

TABLE 39
Process Parameters for Ezetimibe Granulation

| Batch No: 4759-S1-094 | | Batch |
|---|---|---|
| Sr. No. | Process parameters | Details |
| 1. | Binder solution preparation | |
| | Homogenization time | 30 minutes |
| 2. | Granulation process | |
| | Product bed temperature | 30-45° C. |
| 3. | Drying | |
| | Product bed temperature | 45-60° C. |
| | LOD of dried granules | 1.73% (NMT 2% at 105° |
| | Sifting of dried granules | #30 mesh sieve |

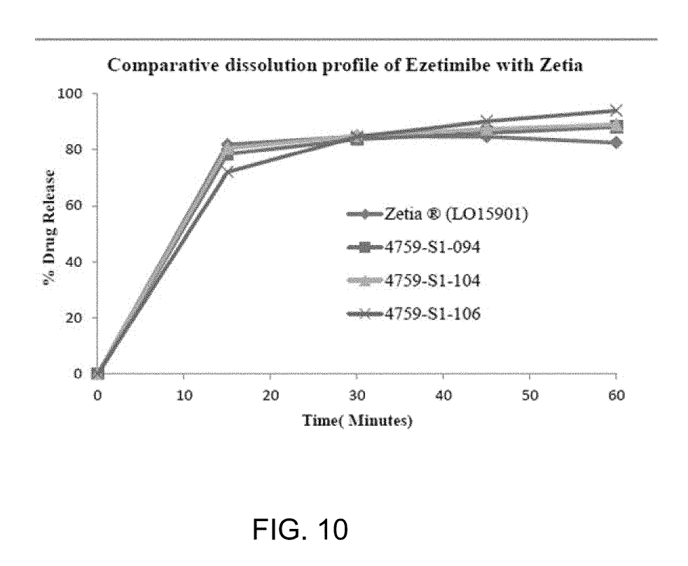

Two reproducibility batches (Batch no: 4759-S1-104 and 4759-S1-106) were manufactured using the same set of parameters. Dissolution profiles obtained from the reproducibility trials are shown in Table 40 and comparative dissolution profile is shown in FIG. 10.

TABLE 40
Dissolution Profile for Process Reproducibility Trials

| Time | Zetia (10 mg) | 4759-S1- | 4759-S1- | 4759-S1- |
|---|---|---|---|---|
| (min) | (LO15901) | 094 | 104 | 106 |
| 0 | 0 | 0 | 0 | 0 |
| 15 | 81.8 | 78.4 | 80.4 | 71.9 |
| 30 | 84.8 | 83.7 | 85.0 | 84.6 |
| 45 | 84.6 | 86.0 | 87.4 | 90.0 |
| 60 | 82.5 | 88.1 | 88.9 | 93.8 |

The reproduced batches showed similar release profiles to the previous batch and reference product. The process can be considered reproducible at laboratory scale.

### Formulation Development Study #4: Coating System Selection

During drug-excipient compatibility studies, Opadry white 85F18422 was found to be incompatible with ezetimibe. This observation was confirmed during stability studies of tablets coated with Opadry white. Assay and impurity profile are given in Table 41.

TABLE 41
Assay and Impurity Profile at Stability Condition

| | | Specified | | | | | |
|---|---|---|---|---|---|---|---|
| | | Impurities | | Single | | | |
| | | (EZT) | | max | | Assay (%) | |
| | | | EZT 21 | un- | Total | | Bem- |
| Batch | | Cyclic | (Keto | specified | impu- | | pedoic |
| No | Condition | Ether | impurity) | impurities | rities | EZT | acid |
| 4490- | Initial | 0.02 | 0.05 | BLOQ | 0.07 | 100.0 | 100.1 |
| S1- | 1M 40° C./ | 0.11 | 0.04 | BLOQ | 0.15 | 96.6 | 99.1 |
| 047 | 75% RH | | | | | | |

ND—Not detected,
BLOQ—Below limit of quantitation

Opadry white (85F18422) is a polyvinyl alcohol (PVA) based coating system with polyethylene glycol (PEG) employed as a plasticizer. The increase in cyclic ether impurity was attributed to either of these materials. Therefore, the following Opadry coating systems were studied for compatibility with ezetimibe. To validate the results, the compressed tablets were coated with these Opadry systems and subjected to a stress study.

PVA & PEG containing Opadry system (Opadry White 88A180040)

PVA based PEG-free Opadry white (Opadry White AMB II 88A180040)

PVA and PEG-free HPMC based Opadry white (Opadry White 03K58821).

The impurity profile post exposure for one week at 600 C in an open container was employed as response for comparative evaluation. The results are provided in Table 42.

TABLE 42
Impurity Profile of Bempedoic Acid and
Ezetimibe with different Opadry systems

| Name of Opadry | | |
|---|---|---|
| | Opadry AMB | |
| Opadry White | II 88A180040 | Opadry White |
| 85F18422 | White | 03K58821 |
| PVA and | PVA (w/o PEG) | HPMC |
| PEG based Opadly | based Opadry) | based Opadry |

| | | Impurity profile | | | | |
|---|---|---|---|---|---|---|
| | | EZT | | EZT 21 | | |
| | | cyclic | RRS | (Keto | | Total |
| Sample | Condition | ether | isomer | impurity) | SMUI | impurities |
| Ezetimibe + | Initial | ND | ND | 0.03 | ND | 0.03 |
| Bempedoic acid + | 2W 60° C. closed | 9.05 | ND | 0.13 | 1.38 | 11.06 |
| Opadry white | 4W 40° C./75% RH | 6.27 | ND | 0.08 | 0.11 | 0.29 |
| (85F18422) | Closed | | | | | |
| Ezetimibe + | Initial | 0.01 | ND | 0.03 | 0.02 | 0.07 |
| Bempedoic acid + | 2W 60° C. closed | 0.02 | ND | 0.04 | 0.02 | 0.14 |
| Opadry White | 4W 40° C./75% RH | 0.03 | ND | 0.04 | 0.02 | 0.11 |
| AMB II | Closed | | | | | |
| 88A180040 | | | | | | |
| Ezetimibe + | Initial | ND | ND | 0.04 | 0.02 | 0.08 |
| Bempedoic acid + | 2W 60° C. closed | ND | ND | 0.04 | 0.02 | 0.08 |
| Opadry White | 4W 40° C./75% RH | ND | ND | 0.05 | 0.03 | 0.10 |
| AMB II | Closed | | | | | |
| 03K58821 | | | | | | |

### Accelerated Stability Data:

Stress stability data of compressed tablets coated with different Opadry system at 60° C. for 1 week is given in Table 43.

TABLE 43
Stress Stability Data of Tablets Coated with
Different Opadry systems at 60° C.

| | 4490-S1-030 | 4490-S1-052A | 4490-S1-052B |
|---|---|---|---|
| | (Opadry white | (Coated with AMB II | (Opadry White |
| Impurity | 88A180040) | 88A180040 White) | 03K58821) |
| EZT (Cyclic ether impurity) | 0.61 | 0.02 | 0.01 |
| EZT (Keto impurity) | 0.07 | 0.04 | 0.04 |
| Single max unspecified impurity | 0.06 | BLOQ | BLOQ |
| Total impurities | 0.74 | 0.06 | 0.04 |

The study confirmed previous observations of drug instability with Opadry White 88A180040. The alternate Opadry systems, Opadry White AMB II 88A180040 and Opadry White 03K58821, were found to be compatible with the APIs as reflected by the level of ezetimibe cyclic ether impurity. These results were confirmed during the stress stability studies on the coated tablet formulation. Among the two Opadry systems; Opadry White was AMB II 88A180040 selected as it did not contain the polyethylene glycol and exhibited relatively superior processability.

### 6.2.3 Updated Risk Assessment of Drug Substance Attributes 6.2.3

Table 44 and Table 45 summarize the updated risk assessment of the drug substance attributes of bempedoic acid and ezetimibe.

TABLE 44
Bempedoic Acid Updated Risk Assessment
& Justification of the Drug Substance Attributes

| Drug | Drug | | |
|---|---|---|---|
| substance | Products | Updated | |
| attributes | CQAs | risk | Justification |
| Particle Size | Dissolution | Low* | The impact of change in particle size of the drug substance |
| Distribution | | | upon the dissolution profile is known (dissolution study with |
| | | | coarser and fine grade API). Control of the drug substance |
| | | | particle size is critical. The risk was reduced from medium to |
| | | | low. |
| Solubility | Dissolution | Low* | Bempedoic acid shows pH dependent dissolution profile. |
| | | | Solubility increases at higher pH. In QC media (phosphate |
| | | | buffer pH 6.8) formulation shows comparable release profile |
| | | | with reference product. The risk was reduced from medium to |
| | | | low |
| Flow | Assay | Low* | Granulation process improves the flow of bempedoic acid |
| Properties | Content | | which reduces the risk related to the assay and content |
| | | | uniformity during processing. The risk was reduced from |
| | | | medium to low |
| Total | Degradation | Low* | Controlled impurity level in API & also no unspecified |
| impurity | product | | impurity generated in the formulation. The risk was reduced |
| | | | from medium to low. |

*For validation with multiple API batches

TABLE 45
Ezetimibe Updated Risk Assessment &
Justification of the Drug Substance Attributes

| Drug | Drug | | |
|---|---|---|---|
| substance | Products | Updated | |
| attributes | CQAs | risk | Justification |
| Particle Size | Assay | Low* | Using top spray wet granulation gave size enlargement and |
| Distribution | CU | | improved flow significantly with satisfactory assay & CU. |
| (PSD) | | | The risk was reduced from medium to low. |
| | Dissolution | | Micronized API was selected from IEVA. Homogenization |
| | | | was adapted to improve dissolution of ezetimibe. The risk was |
| | | | reduced from high to low. |
| Solubility | Dissolution | Low* | 2.0 mg SLS per tablet was used with homogenization prior to |
| | | | top spray granulation process. The risk was reduced from |
| | | | medium to low. |
| Chemical | Degradation | Low* | Impurity levels are controlled in API & taken necessary |
| Stability | product | | precaution during formulation to reduce the level of |
| | | | degradation. The risk was reduced from medium to low. |
| Flow | Assay | Low* | Wet granulation approach improves the flow with satisfactory |
| Properties | CU | | assay and CU. The risk was reduced from medium to low. |
| Process | Degradation | Low* | Process impurity of drug substance was controlled in API and |
| impurity | product | | selected compatible excipients in the formulation to reduce the |
| | | | degradation. Also monitored during stability. The risk is |
| | | | reduced from medium to low. |

*For validation with multiple API batches

### 6.2.4 Updated Risk Assessment of the Formulation Components:

Acceptable ranges for the high risk formulation variables have been established and were included in the control strategy. Based on the results of the formulation development studies, the risk assessment of the formulation variables was updated in Table 46.

TABLE 46
Updated Formulation Risk Assessment & Justification

| Drug | Drug | | |
|---|---|---|---|
| substance | Products | Updated | |
| attributes | CQAs | risk | Justification |
| Ezetimibe | Dissolution | Low* | From the reverse engineering of Zetia, the selected |
| granules | | | concentration of SLS, 2.0 mg/tablets, showed satisfactory |
| (Surfactant | | | dissolution. The risk was reduced from high to low. |
| concentration) | | | |
| Ezetimibe | Dissolution | Low* | From the trials with varying concentration of binder, the |
| granules | | | selected concentration of povidone, 1.0 mg/tablets, shows |
| (Binder | | | satisfactory dissolution. The risk was reduced from medium |
| concentration) | | | to low. |
| Coating | Degradation | Low* | From the compatibility study, the selected PEG free Opadry |
| system | product | | system Opacity AMB II 88A180040 White shows significantly |
| | | | lower degradation as compared to Opadry with PEG. The risk |
| | | | was reduced from medium to low. |

*For validation with multiple API batches

### 6.3 Manufacturing Process Development

Two approaches were used for manufacturing of FDC tablets: a monolayer and a bilayer. Batches produced where put on a stability study.

The monolayer process included manufacturing ezetimibe and bempedoic acid granules separately, blending them together, and compressing to a single layer tablet. The tablet was then coated.

The bilayer approach involved blending bempedoic acid granules with extra-granular excipients and blending ezetimibe granules with extragranular excipients. The two lubricated blends were s compressed into a two layered FDC tablet, one layer comprising bempedoic acid and the other comprising ezetimibe. The bilayer FDC tablets were then coated.

### 6.3.1 FDC-Monolayer Tablet:

Table 47 provides the composition of the monolayer tablet.

TABLE 47
Composition Details of Optimized Formulation for FDC-Monolayer Product

| | |
|---|---|
| Ingredients | mg/unit |

Ezetimibe granulation (Batch no: 4759-S1-094 (Batch size: 2000 units)

| | |
|---|---|
| Ezetimibe | 10.00 |
| Lactose monohydrate (Pharmatose ® 200M) | 50.00 |
| Microcrystalline cellulose (Avicel ® PH-102) | 11.00 |
| Sodium starch glycolate (Primojel ®) | 6.00 |
| Kollidone ® 30 (povidone K30) | 1.00 |
| Sodium lauryl sulfate (Kolliphor SLS Fine) | 2.00 |
| Purified water | q.s. |
| Total weight of granules | 80.00 |

Bempedoic acid granulation (Batch no: 4490-S1-093) (Batch size: 2000 units)

| | |
|---|---|
| Bempedoic acid | 180.00 |
| Colloidal silicon dioxide (Aerosil ® 200P) | 3.50 |
| Microcrystalline cellulose (Avicel ® PH-102) | 9.60 |
| Hydroxypropyl cellulose (HPC-L) | 12.00 |
| Total weight of bempedoic acid granules | 205.10 |

Extra granular excipients (Batch no: 4490-S1-096) (Batch size: 1000 units)

| | |
|---|---|
| Ezetimibe granules | 80.00 |
| Bempedoic acid granules | 205.1 |
| Sodium starch glycolate (Primojel ®) | 21.00 |
| Lactose monohydrate (Pharmatose ® 200M) | 25.00 |
| Microcrystalline cellulose (Avicel ® PH-102) | 40.00 |
| Colloidal silicon dioxide (Aerosil ® 200P) | 0.50 |
| Magnesium stearate | 4.00 |
| Total weight of core tablets | 375.60 |
| Opadry ABMII white 88A180040 | 9.40 |
| Weight of coated tablet | 385.00 |

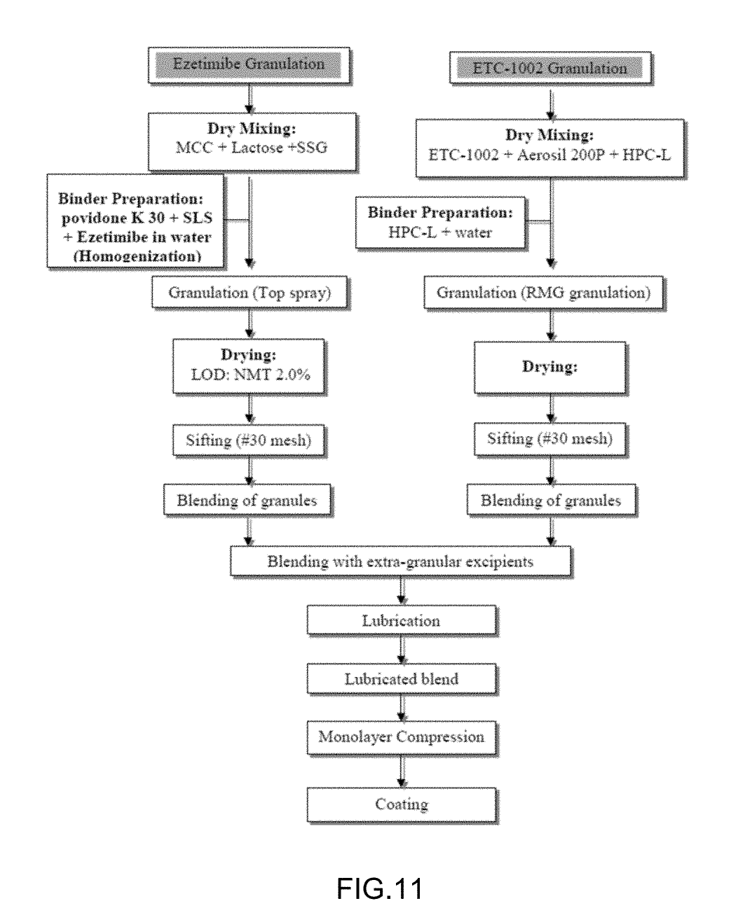

The FDC-monolayer tablet manufacturing process is shown in FIG. 11.

### 6.3.2 FDC-Bilayer Tablet

The composition of final formulation of bilayer tablet is presented in Table 48.

TABLE 48
Bilayer FDC Product Composition

| Ingredients | mg/unit |
|---|---|
| Ezetimibe granulation (Batch no: 4759-S1-104 | |
| (Batch size: 2000 units) | |
| Ezetimibe | 10.00 |
| Lactose monohydrate (Pharmatose ® 200M) | 50.00 |
| Microcrystalline cellulose (Avicel ® PH-102) | 11.00 |
| Sodium starch glycolate (Primojel ®) | 6.00 |
| Kollidone ® 30 (povidone K30) | 1.00 |
| Sodium lauryl sulfate (Kolliphor ® SLS) | 2.00 |
| Microcrystalline cellulose (Avicel ® PH-102) | 9.00 |
| Magnesium stearate | 1.00 |
| Purified water | q.s. |
| Total weight of lubricated blend | 90.00 |
| Bempedoic acid granulation (Batch no: 4759-S1-101) | |
| (Batch size: 2000 units) | |
| Bempedoic acid | 180.00 |
| Colloidal silicon dioxide (Aerosil ® 200P) | 3.50 |
| Microcrystalline cellulose (Avicel ® PH-102) | 09.60 |
| Hydroxypropyl cellulose (HPC-L) | 12.00 |
| Sodium starch glycolate (Primojel ®) | 21.00 |
| Lactose monohydrate (Pharmatose ® 200M) | 25.00 |
| Microcrystalline cellulose (Avicel ® PH-102) | 31.00 |
| Colloidal silicon dioxide (Aerosil ® 200P) | 0.50 |
| Magnesium stearate | 3.00 |
| Total weight of lubricated blend | 285.6 |
| Component of Bilayer compression: (Batch no: 4759-S1-111) | |
| (Batch size: 1000 units) | |
| Ezetimibe lubricated blend | 90.00 |
| Bempedoic acid lubricated blend | 285.6 |
| Total weight of core tablets | 375.60 |
| Opadry ABMII white 88A180040 | 9.40 |
| Weight of coated tablet | 385.00 |

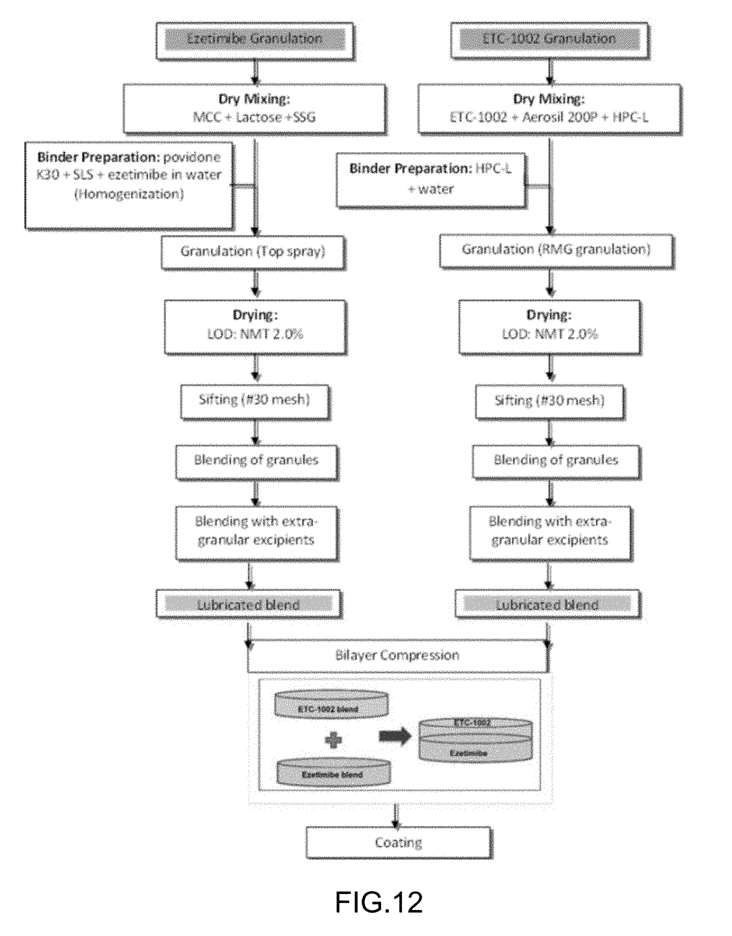

The FDC-bilayer tablet manufacturing process is depicted in FIG. 12.

### Physical Parameters of Compressed Tablets

Table 49 includes the compression parameters for both tablet variants:

TABLE 49
Compression Parameters of FDC Tablets

| | FDC-Monolayer | FDC-Bilayer |
|---|---|---|
| | tablet | tablet |
| Batch Number | 4490-S1-096 | 4759-S1-111 |
| Individual tablet weight (mg) | 368-380 | 373-380 |
| Hardness (N) | 80-90 | 80-110 |
| Thickness (mm) | 4.50-4.55 | 4.31-4.38 |
| Friability (%) | 0.20 | 0.18 |
| Disintegration time (min) | 2 to 4 minutes | 3 to 5 minutes |

### Observations:

Physical parameters of lubricated blend and compression tablets were found to be satisfactory for both variants.

Coating parameters were found to be satisfactory.

### 6.3.3 Dissolution of FDC Formulations in Discriminatory Dissolution Media:

Dissolution profile for ezetimibe in discriminatory dissolution media:

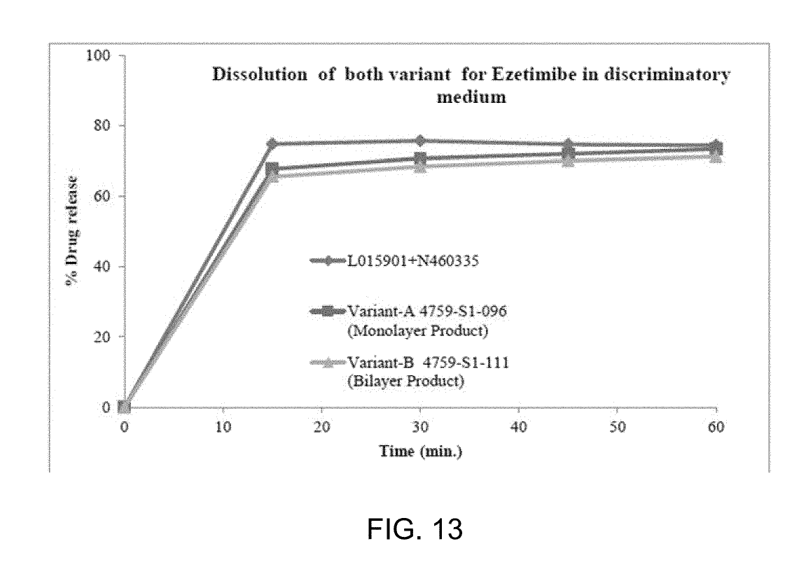

Comparative dissolution profile of ezetimibe from both variants of FDC product vs. Zetia (10 mg)+Bempedoic acid (180 mg) is shown in Table 50 and FIG. 13.

TABLE 50
Ezetimibe release from monolayer and bilayer FDC in discriminatory media
Dissolution comparison with Zetia (10 mg)
Dissolution condition: 0.1% SLS in 0.05M acetate buffer,
pH 4.5 USP Apparatus-II, 50 rpm, 900 mL

| | Zetia (10 mg) + | | |
|---|---|---|---|
| | Bempedoic acid | FDC Product | |
| | (180 mg) | FDC-Monolayer | FDC-Bilayer |
| Product | (Reference Product) | Tablet | Tablet |
| Batch/Lot No. | L015901 + N460335 | 4759-S1-096 | 4759-S1-111 |
| 15 | 74.7 | 67.5 | 65.5 |
| 30 | 75.6 | 70.5 | 68.4 |
| 45 | 74.6 | 71.7 | 70.0 |
| 60 | 74.3 | 73.1 | 71.2 |

### Conclusion:

The dissolutions of both the monolayer and bilayer FDC tablets were found to be comparable to the dissolution Zetia (10 mg) (ezetimibe)+bempedoic acid (180 mg) (reference product).

Dissolution Profile for Bempedoic Acid in Discriminatory Dissolution Medium:

The QC release media, phosphate buffer, pH 6.8, exhibited dose dumping (almost above 90% in 15 minutes). Based on the optimization of surfactant concentration (0.1% to 0.45%) and dissolution volume (500 mL to 1000 mL), 0.45% SLS in acetate buffer, pH 4.5, 1000 mL, 50 rpm, paddle (USP App-II) gave gradual release profile as compared to QC media.

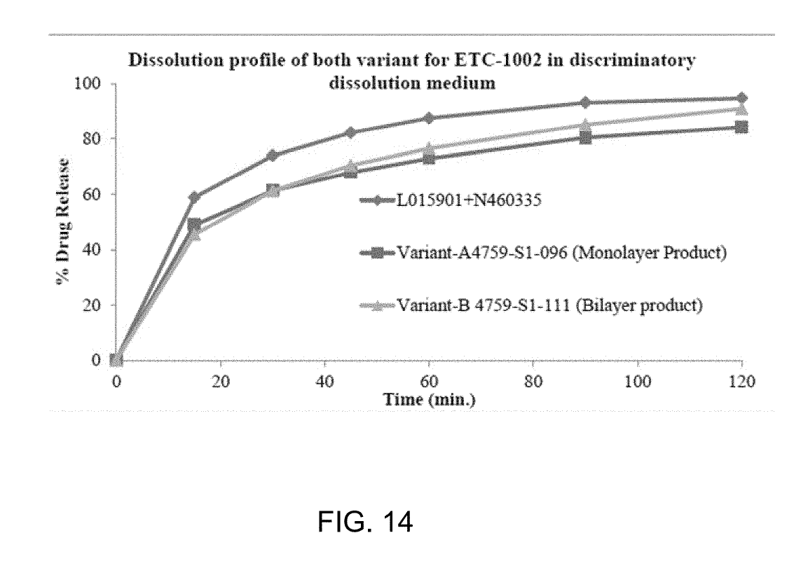

Table 51 and FIG. 14 show comparative dissolution profiles of Bempedoic acid from both variates of the FDC product vs Zetia (10 mg)+bempedoic acid (180 mg) (reference product).

TABLE 51
Dissolution Profile for Bempedoic Acid in Discriminatory Dissolution Media
Dissolution comparison for BEMPEDOIC ACID
Dissolution condition: 0.45% SLS in acetate buffer, pH 4.5, 1000 mL,
50 rpm, paddle (USP App-II)

| | Zetia (10 mg) + | | |
|---|---|---|---|
| | Bempedoic acid (180 mg) | FDC Product | |
| Product | (Reference Product) | Monolayer | Bilayer |
| Batch No. | L015901 + N460335 | (4759-S1-096) | (4759-S1411) |
| Time (min.) | % Drug Release (Bempedoic acid) | | |
| 15 | 58.8 | 48.9 | 45.4 |
| 30 | 73.9 | 6L2 | 61.1 |
| 45 | 82.2 | 67.8 | 70.4 |
| 60 | 87.5 | 72.9 | 76.5 |
| 90 | 93.1 | 80.4 | 85.1 |
| 120 | 94.7 | 84.2 | 90.9 |

### Conclusion:

The % drug release of BEMPEDOIC ACID from both variants of the FDC exhibit release profiles comparable to the reference product.

Dissolution Profile of FDC Prototype Product (Ezetimibe Component) in QC Dissolution medium:

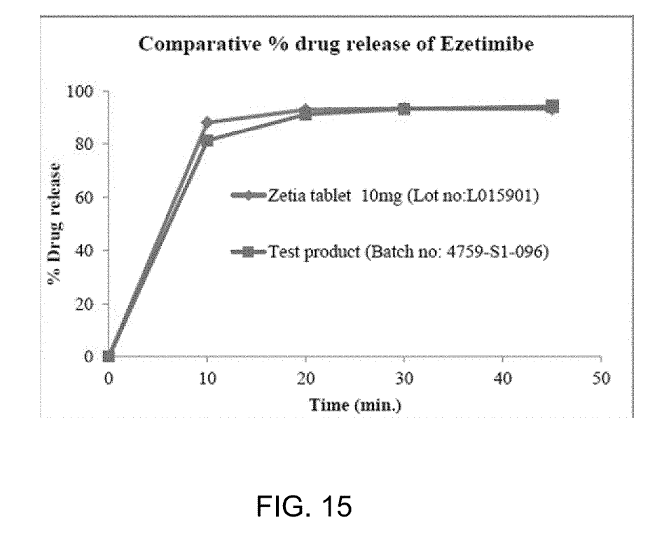

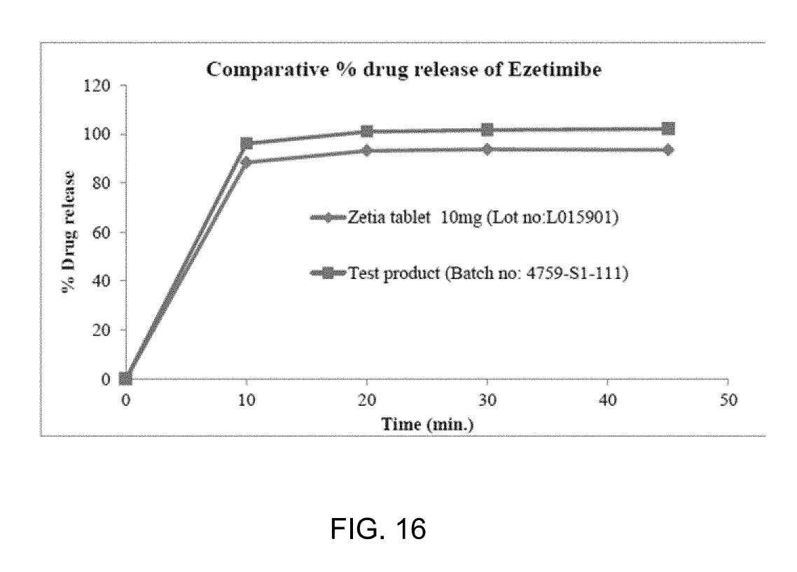

Dissolution Profile of FDC test product (ezetimibe component) in QC media is given in Table 52, Table 53, FIG. 15, and FIG. 16.

TABLE 52
Dissolution Profile of Ezetimibe Component from FDC-Monolayer Tablet
(Batch No: 4759-S1-096)
Dissolution condition
0.45% SLS in acetate buffer, pH 4.5, USP Apparatus-II, 50 rpm, 500 mL

| Product | Zetia (10 mg) | Prototype Product |
|---|---|---|
| Batch No. | L015901 | 4759-S1-096 |
| Time (min.) | % Drug Release (ezetimibe) | |
| 10 | 88.4 | 81.5 |
| 20 | 93.2 | 91.5 |
| 30 | 93.7 | 93.5 |
| 45 | 93.5 | 94.4 |

TABLE 53
Dissolution Profile of Ezetimibe Component of FDC-Bilayer Tablet
(Batch no: 4759-S1-111)
Dissolution condition
0.45% SLS in 0.05M acetate buffer, pH 4.5, USP Apparatus-II,
50 rpm, 500 mL

| Product | Zetia (10 mg) | Prototype Product |
|---|---|---|
| Batch No. | L015901 | 4759-S1-111 |
| Time (min.) | % Drug Release (Ezetimibe) | |
| 10 | 88.4 | 96.1 |
| 20 | 93.2 | 101.0 |
| 30 | 93.7 | 101.7 |
| 45 | 93.5 | 102.1 |

### Observation:

Dissolution of both the variants (monolayer and bilayer) showed similar dissolution to the reference product, Zetia (10 mg) in QC media.

Dissolution profile of FDC test product (bempedoic acid component) in QC media:

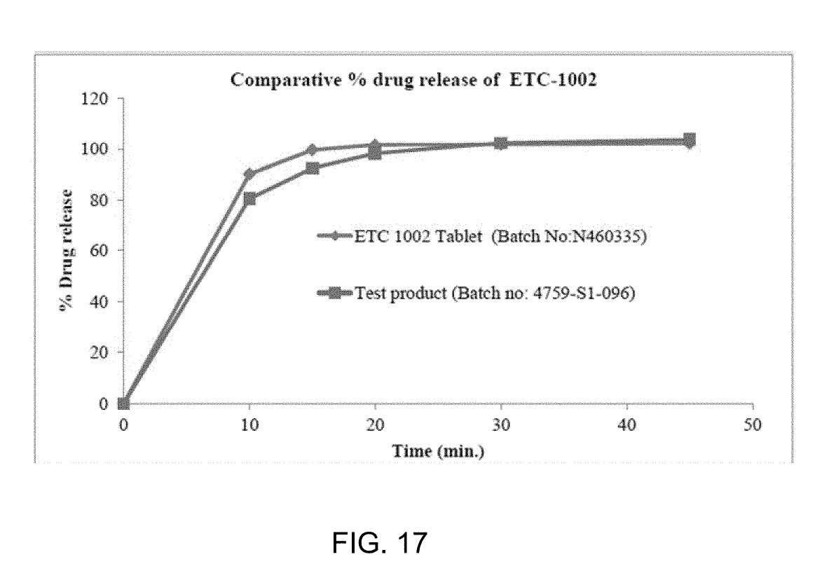

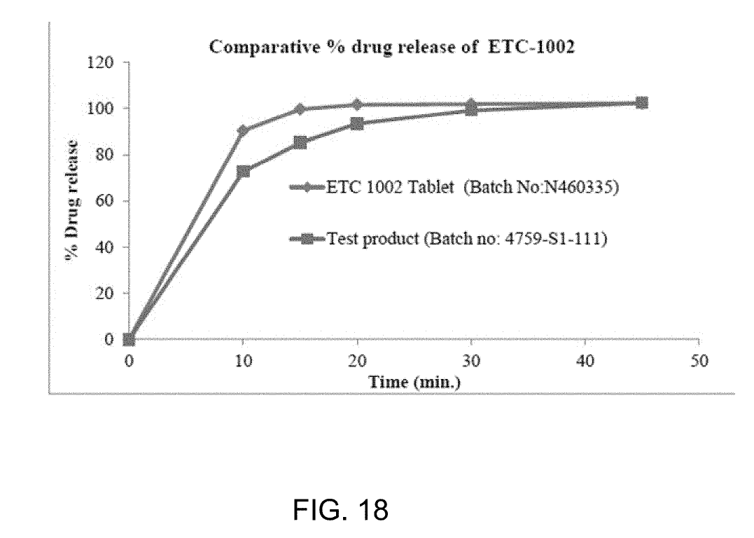

Dissolution profile of FDC test product (bempedoic acid component) is given in Table 54, Table 55, FIG. 17, and FIG. 18.

TABLE 54
Dissolution Profile of Bempedoic Acid Component of
FDC-Monolayer Tablet
(Batch No: 4759-S1-096)
Dissolution condition
50 mM phosphate buffer, pH 6.8, USP Apparatus-II, 50 rpm, 900 mL

| | Bempedoic acid (180 mg) | FDC-Monolayer |
|---|---|---|
| Product | Reference Product | Test product |
| Batch No. | N460335 | 4759-S1-096 |
| Time (min.) | % Drug Release (Bempedoic acid) | |
| 10 | 90.2 | 80.5 |
| 15 | 99.6 | 92.5 |
| 20 | 101.6 | 98.2 |
| 30 | 101.8 | 102.4 |
| 45 | 102.2 | 103.7 |

TABLE 55
Dissolution Profile of Bempedoic Acid Component of FDC-Bilayer Tablet
(Batch no: 4759-S1-111)
Dissolution condition
50 mM phosphate buffer, pH 6.8, USP Apparatus-II, 50 rpm, 900 mL

| | Bempedoic acid (180 mg) | BFDC-Bilayer |
|---|---|---|
| Product | Reference Product | Test product |
| Batch No. | N460335 | 4759-S1-111 |
| Time (min.) | % Drug Release (Bempedoic acid) | |
| 10 | 90.2 | 72.5 |
| 15 | 99.6 | 85.1 |
| 20 | 101.6 | 93.2 |
| 30 | 101.8 | 99.1 |
| 45 | 102.2 | 102.4 |

### Observation:

Dissolution of bempedoic acid from both variants showed similar dissolution profiles to the reference product in QC medium.

### 6.4 Stability Study

For stability loading, 30 tablets were packed in 30 cc HDPE bottles with 1 g polyester coil and 1 g sorbit canister. The bottles were sealed with 28 mm CRC caps.

### 6.4.1 Stability Data of FDC-Monolayer Variant:

Stability data for the monolayer tablets is given in Table 56.

TABLE 56
Stability Data of FDC-Monolayer Tablets (Batch no: 4759-S1-096)

| | | | | Condition: 40°/75% RH | |
|---|---|---|---|---|---|
| Sr. No. | Test | | Initial | 1M | 2M |
| 01 | Physical appearance | | White colored, oval | Complies | |
| | | | shaped film coated | | |
| | | | tablet debossed “000” | | |
| | | | on one side and | | |
| | | | “ABC” on other side | | |
| 02 | Disintegration time (min) | | 2-3 minutes | 3-4 minutes | 2-3 minutes |
| 03 | Hardness (N) | | 98-100 | 100-123 | 103-112 |
| 04 | Water by KF (% w/v) | | 2.96 | 2.65 | 2.45 |
| 03 | Assay (%) | Ezetimibe | 103.2 | 104.0 | 104.9 |
| | | Bempedoic acid | 103.9 | 100.0 | 100.0 |
| 04 | Impurity | Ezetimibe | | | |
| | | Cyclic Ether | ND | BLOQ | BLOQ |
| | | EZT 21(Keto | BLOQ | BLOQ | 0.09 |
| | | impurity) | | | |
| | | Single max | BLOQ | BLOQ | BLOQ |
| | | unspecified | | | |
| | | impurities | | | |
| | | Total impurities | BLOQ | BLOQ | 0.09 |
| | | Bempedoic acid | | | |
| | | Unspecified | ND | ND | 0.06 |
| | | impurity | | | |
| 05 | Dissolution | Time (min) | | | |

| | | | | |
|---|---|---|---|---|
| For ezetimibe: | 10 | 81.5 | 85.8 | 71.1 |
| 0.45% SLS in 0.05M | 20 | 91.5 | 94.0 | 95.4 |
| acetate buffer, pH 4.5, | 30 | 93.5 | 95.8 | 100.6 |
| USP Apparatus-II, | 45 | 94.4 | 96.8 | 102.1 |
| 50 rpm, 500 ml | | | | |
| | Time (min) | | | |
| For bempedoic acid: | 10 | 84.9 | 75.3 | 77.0 |
| 50 mM phosphate buffer, | 20 | 100.8 | 93.4 | 95.2 |
| pH 6.8, USP Apparatus-II, | 30 | 104.8 | 99.3 | 100.5 |
| 50 rpm, 900 ml | 45 | 106.5 | 102.2 | 104.1 |

BLOQ—Below limit of quantification <0.05%, ND—Not detected

### 6.4.2 Stability Data of FDC-Bilayer Variant:

Stability data for the bilayer tablet is given in Table 57.

TABLE 57
Stability Data of FDC-Bilayer Tablet (Batch no: 4759-51-111)

| | | | | Condition: 40° C./75% RH | |
|---|---|---|---|---|---|
| Sr. No. | Test | | Initial | 1M | 2M |
| 01 | Physical appearance | | White colored, oval | Complies | |
| | | | shaped film coated | | |
| | | | tablet debossed “000” | | |
| | | | on one side and | | |
| | | | “ABC” on other side | | |
| 02 | Disintegration time (min) | | 4-5 minutes | 5-6 minutes | 2-5 minutes |
| 03 | Hardness (N) | | 119-122 | 101-150 | 126-141N |
| 04 | Water by KF (% w/v) | | 2.71 | 2.61 | 2.51 |
| 05 | Assay (%) | Ezetimibe | 99.1 | 101.8 | 103.2 |
| | | Bempedoic acid | 102.5 | 97.5 | 97.1 |
| 06 | Impurity | Ezetimibe | | | |
| | | Cyclic Ether | ND | BLOQ | BLOQ |
| | | EZT 21(Keto | BLOQ | BLOQ | 0.09 |
| | | impurity) | | | |
| | | Single max | ND | BLOQ | BLOQ |
| | | unspecified | | | |
| | | impurities | | | |
| | | Total impurities | BLOQ | BLOQ | 0.09 |
| | | Bempedoic acid | | | |
| | | Unspecified | ND | ND | 0.05 |
| | | impurity | | | |
| 07 | Dissolution | Time (min) | | | |

| | | | | |
|---|---|---|---|---|
| For Ezetimibe: | 10 | 96.1 | 91.8 | 93.7 |
| 0.45% SLS in 0.05M | 20 | 101.0 | 99.1 | 98.6 |
| acetate buffer, pH 4.5, | 30 | 101.7 | 100.4 | 99.4 |
| USP Apparatus-II, | 45 | 102.1 | 101.3 | 99.9 |
| 50 rpm, 500 ml | | | | |
| | Time (min) | | | |
| For bempedoic acid: | 10 | 72.5 | 63.3 | 71.5 |
| 50 mM phosphate buffer, | 20 | 93.2 | 88.4 | 88.2 |
| pH 6.8, USP Apparatus-II, | 30 | 99.1 | 97.5 | 94.0 |
| 50 rpm, 900 ml | 45 | 102.4 | 103.0 | 96.5 |

BLOQ—Below limit of quantification <0.05%, ND—Not detected

### Conclusion:

All physicochemical parameters were found to be satisfactory and comparable with the initial at 2 months under accelerated conditions.

Dissolution in QC media was found to be comparable (>90% release in 30 min).

Less than 0.1% unspecified impurity of bempedoic acid was generated at 2 months under accelerated condition.

Impurity profiling was found to be within specification at 2 months under accelerated conditions in accordance with ICH Q3B (R2) based on the maximum daily dose.

### LIST OF ABBREVIATIONS

| | | | |
|---|---|---|---|
| ACL | ATP citrate lyase | MCC | Microcrystalline cellulose |
| BCS | Biopharmaceutics Classification System | mg | Milligram |
| BLOQ | Below level of quantification | Min | Minute |
| cfu | Colony forming unit | mL | Milliliter |
| CQA | Critical quality attribute | MLT | Microbial Limit Test |
| CR | Child Resistant | mm | Millimeter |
| CU | Content uniformity | mM | Millimolar |
| DMF | Drug master file | mPa · s | Millipascal · second |
| DMSO | Dimethyl sulfoxide | N | Newton |
| EZT | Ezetimibe | ND | Not detected |
| FBP | Fluid Bed Processer | NDA | New drug application |
| FDC | Fixed dose combination | NF | National formulary |
| g/mol | gram per mole | NMT | Not more than |
| GMP | Good manufacturing practice | PDR | Pharmaceutical Development |
| | | | Report |
| HDPE | High density polyethylene | PEG | Polyethylene glycol |
| HPC-L | Hydroxypropyl cellulose low substituted | PI | Purity Index |
| HPLC | High performance liquid chromatography | PK | Pharmacokinetic |
| HPMC | Hydroxypropyl methyl cellulose | PSD | Particle size distribution |
| hrs | Hours | PVA | Polyvinyl alcohol |
| ICH | International Conference on Harmonization | QbD | Quality by design |
| IH | In house | QL | Quantification limit |
| IID: | Inactive ingredient database | QTPP | Quality target product profile |
| IR | Immediate release | RH | Relative humidity |
| LDL-C | Low-density lipoprotein cholesterol | RLD | Reference listed drug |
| RMG | Rapid mixer granulator | TAMC | Total Aerobic Microbial |
| | | | Count |
| RPM | Rotation per minute | TYMC | Total Yeast and Mold Count |
| SLS | Sodium lauryl sulfate | USP | United States Pharmacopeia |
| SSG | Sodium starch glycolate | XRD | X-ray diffraction |

### Example 5

ETC 1002 Study for Resolving Problem of Sticking

Laboratory Trials

TABLE 58
Formula:

| | 4490-S1-024 | 4759-S1-058 |
|---|---|---|
| Ingredients | mg/unit | mg/unit |
| ETC 1002 (Bempedoic acid) | 180 | 180 |
| Colloidal silicon dioxide | — | 3 |
| Lactose monohydrate | 30 | — |
| Microcrystalline cellulose | 56 | 31 |
| Hydroxypropyl cellulose | 12 | 12 |
| Sodium Starch Glycolate | 21 | 21 |
| Ezetimibe Granules | 70 | 70 |
| colloidal silicon dioxide | 1.5 | 1 |
| Magnesium stearate | 4.5 | 4 |
| Total weight (mg) | 375 | 342.0 |

### Manufacturing Process for Formula I (4490-S1-024):

All ingredients in dry mix were co-sifted and granulated with binder solution and the obtained granules were dried in a fluid bed dryer. The dried granules were blended with Ezetimibe granules along with microcrystalline cellulose, sodium starch glycolate and lubricated. The granules were then compressed into tablets.

### Manufacturing Process for Formula II (4759-S1-058):

ETC 1002 and colloidal silicon dioxide were co-sifted and blended. This blend was then further mixed with microcrystalline cellulose and granulated binder solution. The granules were dried and sifted. The dried granules were blended with Ezetimibe granules along with microcrystalline cellulose, sodium starch glycolate and lubricated. The granules were then compressed into tablets.

TABLE 59
OBSERVATIONS:

| | Observation | |
|---|---|---|
| Sticking on | formula I | formula II |
| Punch | Sticking was observed on punch surface | Comparatively |
| Turret sticking | Sticking was also seen on turret | less sticking |
| Logo | On ABC and 000 embossing sticking | was observed |
| | was observed on curve surface of letters | |

### Conclusion:

The surface treatment of ETC 1002 with colloidal silicon dioxide reduced sticking which was observed during compression with untreated API.

1. A pharmaceutical composition comprising:
180 mg Bempedoic acid;
10 mg Ezetimibe;
4 mg colloidal silicon dioxide;
4 mg magnesium stearate;
12 mg hydroxypropyl cellulose—low substituted;
1 mg Povidone;
75 mg Lactose monohydrate;
2 mg sodium lauryl sulfate;
60.6 mg microcrystalline cellulose; and
27 mg sodium starch glycolate.

2.-4. (canceled)

5. The pharmaceutical composition of claim 1, wherein the composition is in the form of a tablet and further comprises a polyvinyl alcohol (PVA) based coating; and wherein the coating comprises: polyvinyl alcohol (PVA), glycerol monocaprylocaprate type 1, sodium lauryl sulfate, titanium dioxide and talc.

6. (canceled)

7. The pharmaceutical composition of claim 1, wherein the composition has a bulk density of at least 0.25 gm/ml and no more than 0.55 gm/ml.

8. The pharmaceutical composition of claim 1, wherein the composition has a Carr's Index of at least 10 and no more than 30.

9. The pharmaceutical composition of claim 1, wherein the granules of the composition have an angle of repose of at least 200 and no more than 45°.

10. A granulated composition comprising 180 mg Bempedoic acid;
3.5 mg colloidal silicon dioxide;
9.6 mg microcrystalline cellulose; and
12 mg hydroxypropyl cellulose—low substituted.

11.-12. (canceled)

13. The composition of claim 10, wherein the composition has a Carr's Index of at least 10 and no more than 30.

14. The composition of claim 10, wherein the granules of the composition have an angle of repose of at least 200 and no more than 45°.

15. The composition of claim 10, wherein the Bempedoic acid is present in an amount of at least 50% and no more than 95% by weight of the total formulation.

16. (canceled)

17. A bilayer tablet comprising Bempedoic acid and Ezetimibe, wherein the first layer composition comprises:
Ezetimibe granulated with a pharmaceutically acceptable excipient;
and wherein the second layer composition comprises:
Bempedoic acid granulated with colloidal silicon dioxide and a pharmaceutically acceptable excipient; and
wherein the first and second compositions comprise:
180 mg Bempedoic acid;
10 mg Ezetimibe;
4 mg colloidal silicon dioxide;
4 mg magnesium stearate;
12 mg hydroxypropyl cellulose—low substituted;
1 mg Povidone;
75 mg Lactose monohydrate;
2 mg sodium lauryl sulfate;
60.6 mg microcrystalline cellulose; and
27 mg sodium starch glycolate.

18. The bilayer tablet of claim 17, wherein the Friability of the tablet is at least 0.01% and no more than 0.1%.

19. The bilayer tablet of claim 17, wherein the first layer composition is at least 0.1% and no more than 23% by weight of the total tablet and the second layer composition is at least 0.1% and no more than 74% by weight of the total tablet.

20. (canceled)
